SEQUENCE LISTING (10 sequences)

SEQ ID NO: 1 - DNA, 2737 bp, Homo sapiens
  CDS (108)..(2351)
ggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagct

SEQ ID NO: 2 - protein, 748 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 3 - protein, 28 aa, Homo sapiens
RRRRRRRRRKFVRSRRPRTASCALAFVN

SEQ ID NO: 4 - protein, 19 aa, Homo sapiens
KFVRSRRPRTASCALAFVN

SEQ ID NO: 5 - protein, 30 aa, Homo sapiens
KFVRSRRPRTASCALAFVNMLLRLERILRR

SEQ ID NO: 6 - protein, 17 aa, Homo sapiens
RRRRRRRRRSCALAFVN

SEQ ID NO: 7 - protein, 169 aa, Homo sapiens
MGRRFLVTVRIQRAGRPLQERVFLVKFVRSRRPRTASCALAFVNMLLRLERILRRGPHRNPGPGDDDGQRSRSSSSAQLRCRFELRGPHYLLPPGARRSAGRLPGHAGGAARVRGSAGCARCLGSPAARLGPRAGTSRHRAIFAFRWVLFVFRWVVFVYRWERRPDRRA

SEQ ID NO: 8 - protein, 11 aa, Homo sapiens
YGRKKRRQRRR

SEQ ID NO: 9 - protein, 11 aa, Homo sapiens
YARAAARQARA

SEQ ID NO: 10 - protein, 9 aa, Artificial
  Synthetic sequence
RRRRRRRRR

(19) United States
(12) Patent Application Publication
(10) Pub. No.: US 2010/0056441 A1
(43) Pub. Date: Mar. 4, 2010

(54) Method for Inhibiting Angiogenesis

(76) Inventors: Robert H. Costa, Oak Park, IL (US); Jane B. Costa, Oak Park, IL (US); I-Ching Wang, Chicago, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/282,113

(22) PCT Filed: Mar. 19, 2007
(86) PCT No.: PCT/US2007/064300

§ 371 Date: Jun. 19, 2009

Related U.S. Application Data
(60) Provisional application No. 60/783,362, filed on Mar. 17, 2006.
(60) Provisional application No. 60/869,656, filed on Dec. 12, 2006.

(51) Int. Cl. A61K 38/17; A61K 38/10; A61K 38/08

(52) U.S. Cl. 514/12; 514/13; 514/15

(57) ABSTRACT

The invention provides methods for inhibiting angiogenesis in an animal in need thereof. The invention also pro-vides methods for preventing tumor growth and metastasis in an animal comprising inhibiting FoxM1B activity.

This application claims the benefit of priority to U.S. provisional application Ser. No. 60/783,362, filed Mar. 17, 2006, and U.S. provisional application Ser. No. 60/869,656, filed Dec. 12, 2006.

This invention was made with government support under AG21842-02 awarded by the National Institute on Aging, and under DK54687-06 awarded by the National Institute of Diabetes and Digestive and Kidney Diseases. The U.S. government has certain rights in the invention.

### BACKGROUND OF THE INVENTION

1. Field of the Invention

The invention relates to cellular proliferation and the control of cell proliferation in animals. More particularly, the invention relates to angiogenesis related to proliferation of cells and tissues in an animal, especially with regard to pathological proliferation associated with tumorigenesis, both benign and malignant, and other diseases and pathologies of improperly-controlled cell proliferation and inflammatory disorders. Specifically, the invention provides methods for inhibiting angiogenesis by reducing expression or inhibiting gene product function of a mammalian gene, FoxM1B, that is involved in control of cell proliferation.

2. Background of the Related Art

The Forkhead box transcription factors have been implicated in regulating cellular longevity and proliferative capacity. Such studies include a finding of increased longevity in C. elegans bearing a mutant daf-2 gene, which encodes the worm homolog of the insulin/Insulin-like Growth Factor 1 (IGF1) receptor (Lin et al., 1997, Science 278: 1319-1322; Ogg et al., 1997, Nature 389: 994-999). Disruption of the daf-2 gene abolishes insulin-mediated activation of the phosphatidylinositol 3-kinase (PI3K)-protein kinase B/Akt (Akt) signal transduction pathway and prevents inhibition of the forkhead transcription factor daf-16 (corresponding to mammalian homologs FoxO1 or Fkhr; Paradis and Ruvkun, 1998, Genes Dev. 12: 2488-2498). Activation of the PI3K/Akt pathway phosphorylates the C-terminus of the Daf-16 (FoxO1; Fkhr) gene product and mediates its nuclear export into the cytoplasm, thus preventing FoxO1 transcriptional activation of target genes (Biggs et al., 1999, Proc. Natl. Acad. Sci. USA 96: 7421-7426; Brunet et al., 1999, Cell 96: 857-68; Guo et al., 1999, J. Biol. Chem. 274: 17184-17192).

More recent studies of Daf-2⁻ C. elegans mutants have demonstrated that Daf-16 stimulates expression of genes that limit oxidative stress (Barsyte et al., 2001, FASEB J. 15: 627-634; Honda et al., 1999, FASEB J. 13: 1385-1393; Wolkow et al., 2000, Science 290: 147-150) and that the mammalian FoxO1 gene could functionally replace the Daf-16 gene in C. elegans (Lee et al., 2001, Curr. Biol. 11: 1950-1957). In proliferating mammalian cells, the PI3K/Akt signal transduction pathway is essential for G1 to S-phase progression because it prevents transcriptional activity of the FoxO1 and FoxO3 proteins, which stimulate expression of the CDK inhibitor p27^{kip1} gene (Medema et al., 2000, Nature 404: 782-787). Moreover, genetic studies in budding yeast demonstrated that forkhead Fkh1 and Fkh2 proteins are components of a transcription factor complex that regulates expression of genes critical for progression into mitosis (Hollenhorst et al., 2001, Genes Dev. 15: 2445-2456; Koranda et al., 2000, Nature 406: 94-98; Kumar et al., 2000, Curr. Biol. 10: 896-906; Pic et al., 2000, EMBO J. 19: 3750-3761).

The Forkhead Box M1B (FoxM1B or FoxM1) transcription factor (also known as Trident and HFH-11B) is a proliferation-specific transcription factor that shares 39% amino acid homology with the HNF-3 winged helix DNA binding domain. The molecule also contains a potent C-terminal transcriptional activation domain that possesses several phosphorylation sites for M-phase specific kinases as well as PEST sequences that mediate rapid protein degradation (Korver et al., 1997, Nucleic Acids Res. 25: 1715-1719; Korver et al., 1997, Genomics 46: 435-442; Yao et al., 1997, J. Biol. Chem. 272: 19827-19836; Ye et al., 1997, Mol. Cell. Biol. 17: 1626-1641).

In situ hybridization studies have shown that FoxM1B is expressed in embryonic liver, intestine, lung, and renal pelvis (Ye et al., 1997, Mol. Cell. Biol. 17: 1626-1641). In adult tissue, however, FoxM1B is not expressed in postmitotic, differentiated cells of the liver and lung, although it is expressed in proliferating cells of the thymus, testis, small intestine, and colon (Id). FoxM1B expression is reactivated in the liver prior to hepatocyte DNA replication following regeneration induced by partial hepatectomy (Id).

FoxM1B is expressed in several tumor-derived epithelial cell lines and its expression is induced by serum prior to the G₁/S transition (Korver et al., 1997, Nucleic Acids Res. 25: 1715-1719; Korver et al., 1997, Genomics 46: 435-442; Yao et al., 1997, J. Biol. Chem. 272: 19827-19836; Ye et al., 1997, Mol. Cell. Biol. 17: 1626-1641). Consistent with the role of FoxM1B in cell cycle progression, elevated FoxM1B levels are found in numerous actively-proliferating tumor cell lines (Korver et al., 1997, Nucleic Acids Res. 25: 1715-1719; Yao et al., 1997, J. Biol. Chem. 272: 19827-36; Ye et al., 1997, Mol. Cell. Biol. 17: 1626-1641). Increased nuclear staining of FoxM1B was also found in human basal cell carcinomas (Teh et al., 2002, Cancer Res. 62: 4773-80), suggesting that FoxM1B is required for cellular proliferation in human cancers.

These studies and others suggest that FoxM1B plays some role in human cancers. FoxM1B, therefore, is an attractive target for anti-cancer therapies because FoxM1B expression typically declines during normal aging (see co-owned and co-pending U.S. patent application Ser. No. 10/650,609, filed Aug. 28, 2003, Ser. No. 10/809,144, filed Mar. 25, 2004, and Ser. No. 11/150,756, filed Jun. 10, 2005, incorporated by reference herein in their entirety). Thus, FoxM1B can provide a selective target that is more active in tumor cells than in normal cells, particularly terminally-differentiated, aged or aging normal cells that surround a tumor, allowing tumor cells to be treated while minimizing the deleterious side-effects of such compounds on normal cells.

Angiogenesis is an important factor in proliferation and metastasis of various progressive solid tumors. Angiogenesis involves steps of, inter alia, stimulation by vascular endothelial growth factor (VEGF), disengagement of peritheliocyte or decomposition or digestion of extracellular matrix, migration and proliferation of vascular endothelial cells, formation of tubule by endothelial cells, formation of basal membrane, and maturation of blood vessels. During tumorigenesis, new blood vessels are developed to supply oxygen and nutrients to tumors to sustain and encourage tumor growth. In addition, vessels serve as a route for infiltration and metastasis of tumor cells to other tissues. Inhibiting angiogenesis is an attractive therapeutic approach to preventing tumor growth and promoting tumor cell death.

Additionally, angiogenesis is involved in many types of disease or condition other than tumors. Thus, it is desirable to have a medicament inhibiting angiogenesis that is effective in preventive and therapeutic treatment of any proliferation dysregulation associated disorders.

### SUMMARY OF THE INVENTION

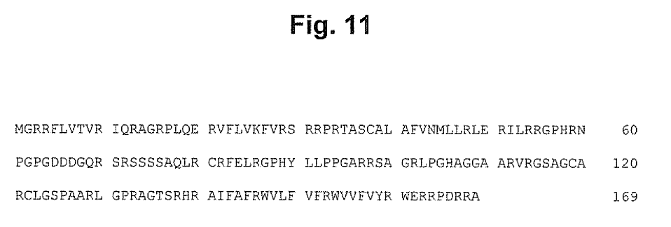

This invention provides methods for inhibiting angiogenesis in a patient in need thereof having a proliferation dysregulation associated disorder. In preferred embodiments, the methods comprise the step of administering to the patient a therapeutically effective amount of a peptide having an amino acid sequence of amino acids 26-44 of the p19^{ARF} tumor suppressor protein as set forth in FIG. 11. Preferably, the peptide is covalently linked to a protein transduction domain (PTD) capable of facilitating peptide entry into cells across the plasma cell membrane. In specific embodiments, the peptide is identified by SEQ ID NO: 3 (rrrrrrrrrKFVRSRRPRTASCALAFVN; referred to herein as the (D-Arg)₉-p 19^{ARF} 26-44 peptide, or WT ARF26-44) or SEQ ID NO: 4 (KFVRSRRPRTASCALAFVN; referred to herein as the p19^{ARF} 26-44 peptide, wherein the peptide of SEQ ID NO: 4 is preferably covalently-linked to a PTD moiety). In a particular aspect, peptides having an amino acid sequence of the p19^{ARF} tumor suppressor protein as set forth in SEQ ID NO: 3 or SEQ ID NO: 4 or SEQ ID NO: 4 covalently linked to a PTD moiety can be used as reagents in the practice of the methods of the invention for preventing or treating diseases in which angiogenesis is involved in causing and/or inducing the onset of the disease. Individuals who would benefit from the practice of the methods of the invention include but are not limited to individuals having diabetic vascular complications, diabetic retinopathy, articular rheumatism, rheumatoid arthritis, diabetes, arteriosclerosis, ulcerative colitis, psoriasis, angiopoietic glaucoma, inflammatory diseases, or benign, malignant or metastatic tumors. In a particular aspect, the invention provides methods for treating hepatocellular carcinoma by inhibiting angiogenesis in a patient, the method comprising administering a peptide, such as a peptide having an amino acid sequence of the p19^{ARF} tumor suppressor protein identified by SEQ ID NO: 3 or SEQ ID NO: 4 or SEQ ID NO: 4 covalently linked to a PTD moiety to said patient.

Specific preferred embodiments of the invention will become evident from the following more detailed description of certain preferred embodiments and the claims.

### DETAILED DESCRIPTION OF THE DRAWINGS

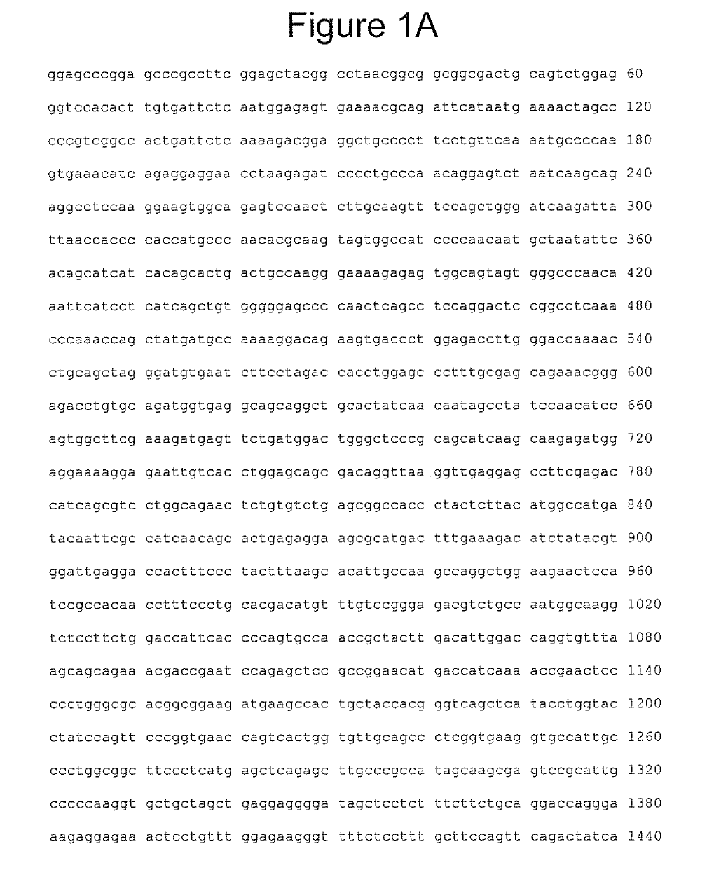

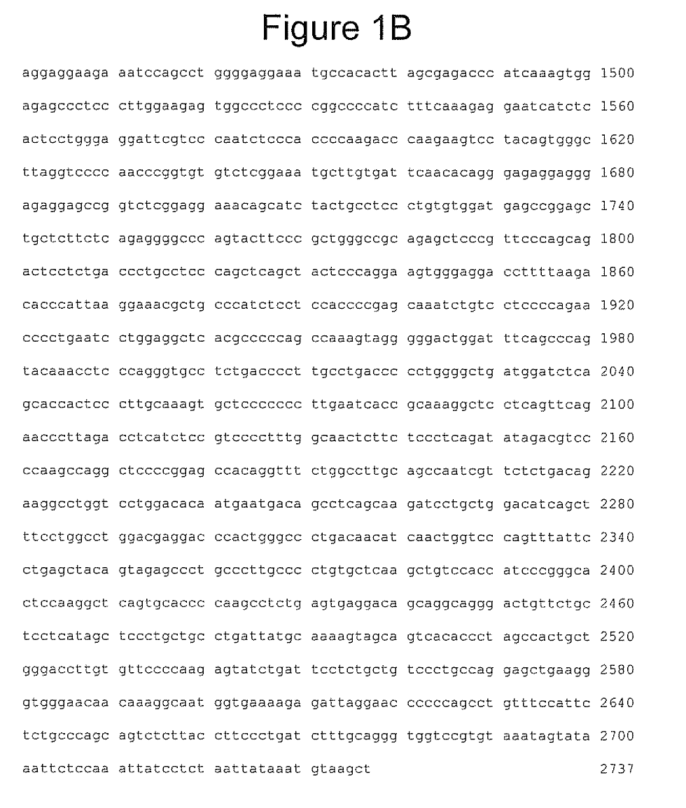

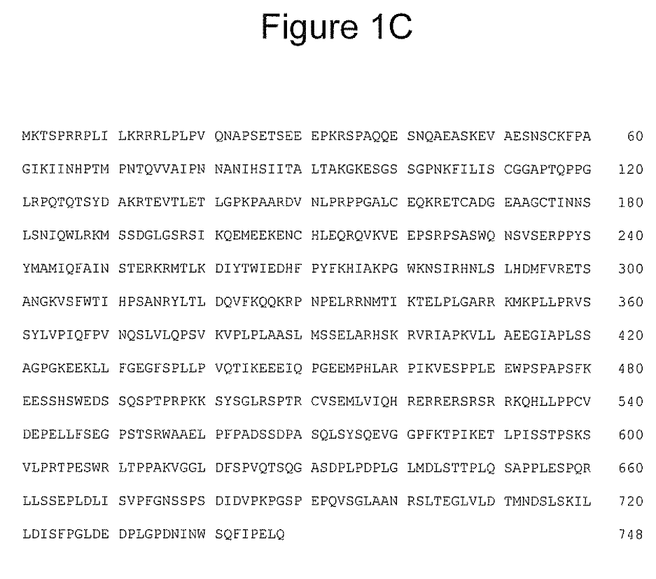

FIGS. 1A and 1B depict a human FoxM1B cDNA comprising a deletion of the terminal 972 nucleotides at the 3′ end of the native molecule (SEQ ID NO: 1).

FIG. 1C depicts a human FoxM1B protein sequence (SEQ ID NO: 2) encoded by the nucleotide sequence as set forth in SEQ ID NO: 1.

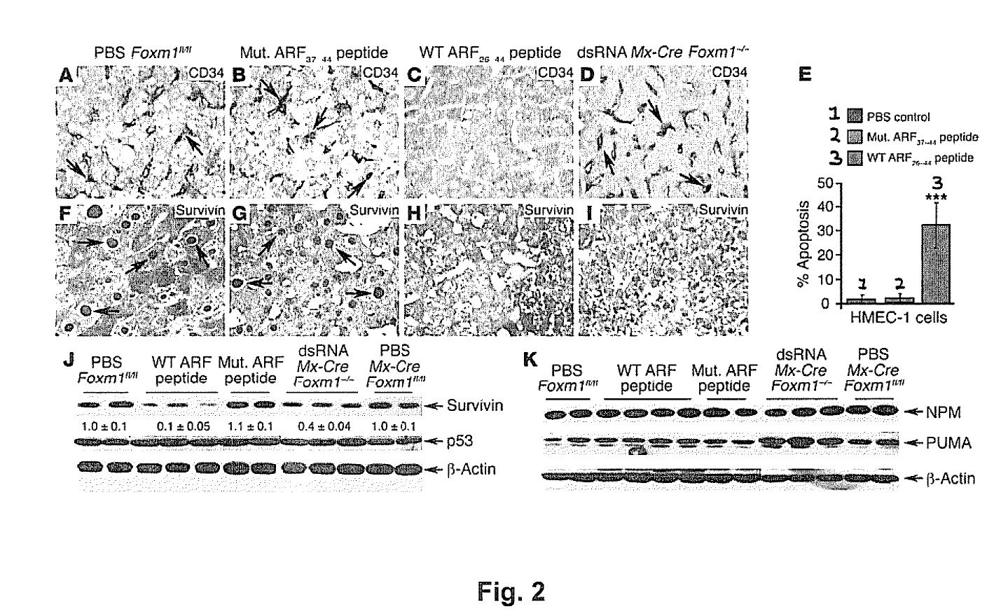

FIGS. 2A through 2G show experimental results demonstrating that WT ARF 26-44 peptide reduced angiogenesis and survival in mouse heptacellular carcinomas (HCC). Specifically, FIGS. 2A-2D are photomicrographs showing CD34 immunostaining of HCC tumor sections from mice treated with an ARF 37-44 peptide (rrrrrrrrrSCALAFVN, SEQ ID NO:6, herein after referred to as “mutant ARF 37-44 peptide”) that has no antiproliferative activity; WT ARF 26-44 peptide, phosphate buffered saline (PBS), or from dsRNA treated Mx-Cre FoxM1 −/− mice (CKO). FIG. 2E is a graphical representation of the results of apoptosis experiments, showing that WT ARF26-44 induced apoptosis in human microvascular endothelial cells (HMEC-1), whereas mutant ARF37-44 peptide or PBS control did not. FIGS. 2F-2I are photomicrographs showing survivin immunostaining of HCC tumor sections of mice treated with mutant ARF 37-44 peptide, WT ARF 26-44 peptide, phosphate buffered saline (PBS), or from dsRNA (CKO) Mx-Cre FoxM1−/− mice. FIG. 2J is an autoradiogram showing Western blot analysis indicating that there was a decrease in survivin protein expression in WT ARF 26-44 peptide treated mouse tumors. FIG. 2K is an autoradiogram showing Western blot analysis indicating that there was no decrease in expression of nucleophosmin protein or p53 regulated pro-apoptotic PUMA protein in WT ARF 26-44 peptide treated mouse tumors.

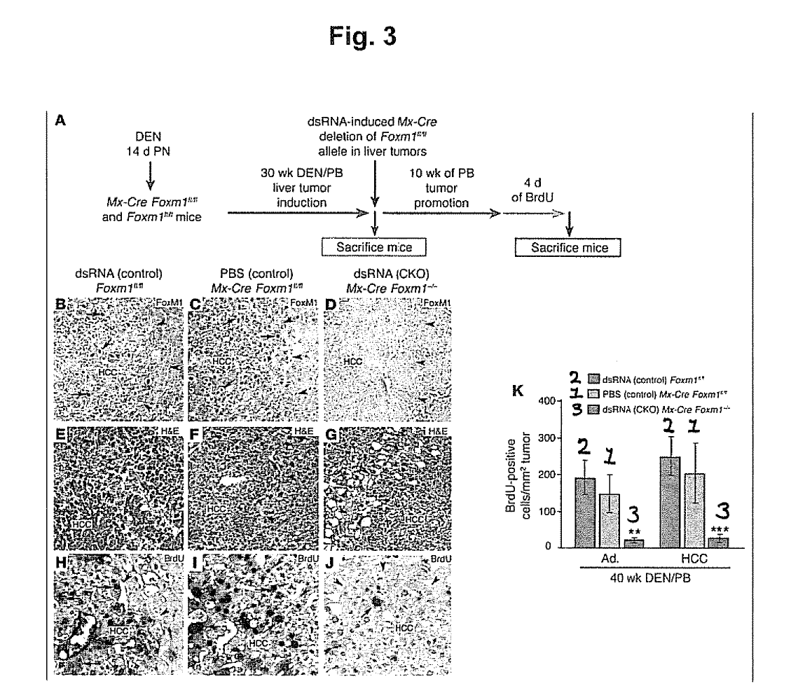

FIGS. 3A through 3K show experimental results demonstrating that the mouse Foxm1 transcription factor is required for hepatic tumor progression. FIG. 3A is a schematic diagram depicting the experimental design of a conditional deletion of Foxm1 f1/f1 mutant in preexisting liver tumors. FIGS. 3B-3D are photomicrographs showing that dsRNA (CKO) Mx-Cre Foxm1 −/− liver tumors displayed no detectable nuclear staining of Foxm1 protein as determined by immunostaining with Foxm1 antibody. FIGS. 3E-3G are photomicrographs showing Hematoxylin and Eosin (H&E) staining of the indicated HCC liver sections after 40 weeks of DEN/PB exposure (tumor margins indicated by arrow heads). FIGS. 3H-3J are photomicrographs showing BrdU incorporation detected by immunostaining of liver tumor sections with monoclonal BrdU antibody from indicated mice at 40 weeks following DEN/PB exposure. Arrows depict nuclear staining for either Foxm1 protein or BrdU. FIG. 3K is a graph showing the mean number of BrdU positive cells per mm² liver tumor (±SD) as described herein. The asterisks indicate statistically significant changes: **P<0.01 and ***P<0.001. Magnification for photomicrographs shown in FIGS. 3B-3G is 200×; for photomicrographs shown in FIGS. 3H-3J it is 400×.

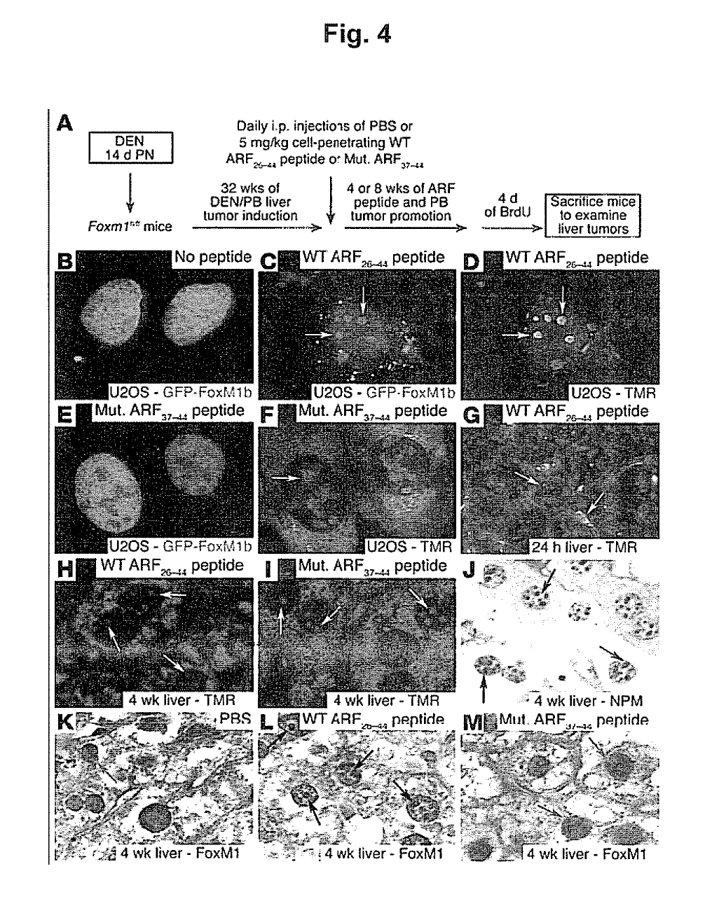

FIGS. 4A through 4M shows experimental evidence that the cell penetrating WT ARF 26-44 peptide targets the liver tumor Foxm1 protein to the nucleolus. FIG. 4A is a schematic diagram showing the experimental design of ARF peptide treatment of liver tumor-bearing mice. Liver tumors were induced in mice with DEN/PB exposure and then subjected to daily intraperitoneal (IP) injections of the cell penetrating WT ARF 26-44 peptide or Mutant ARF 37-44 as described above. FIGS. 4B-4F are photomicrographs showing that GFP-FoxM1b protein was targeted to the nucleolus by the cell penetrating WT ARF 26-44 peptide. U2OS cells were transfected with GFP-FoxM1b expression vector and were either left untreated or incubated for 48 hours with tetramethylrhodamine (TMR) fluorescently-tagged WT ARF 26-44 peptide (shown in the photomicrographs in FIGS. 4C-4D) or mutant ARF 37-44 peptide (FIGS. 4E-4F) and then analyzed for GFP or peptide (TMR) fluorescence. FIG. 4G is a photomicrograph showing TMR WT ARF 26-44 peptide fluorescence localized in the hepatocyte cytoplasm and nucleolus and in the hepatic mesenchymal cells (see arrows). The photomicrographs shown in FIGS. 4H-4I demonstrated that both Mutant ARF 37-44 peptide and WT ARF 26-44 peptide were targeted to the hepatocyte cytoplasm and nucleolus (white arrow) as determined by laser confocal microscopy. FIG. 4J is a photomicrograph showing immunostaining of tumor sections with antibody specific to either nucleolar nucleophosmin (NPM) protein (black arrow) or FoxM1 protein (FIGS. 4K-4M). FIG. 4K is a photomicrograph showing that WT ARF 26-44 peptide targeted FoxM1 in tumor cells to the nucleolus (black arrow, 4L), whereas FoxM1 remained nuclear after treatment with Mutant ARF 37-44 peptide (4M) or PBS (4K). Magnification for the photomicrographs shown in FIGS. 4B-4F and FIGS. 4J-4M is 400×; for FIG. 4G, magnification is 200× and for photomicrographs shown in FIGS. 4H-I it is 600×.

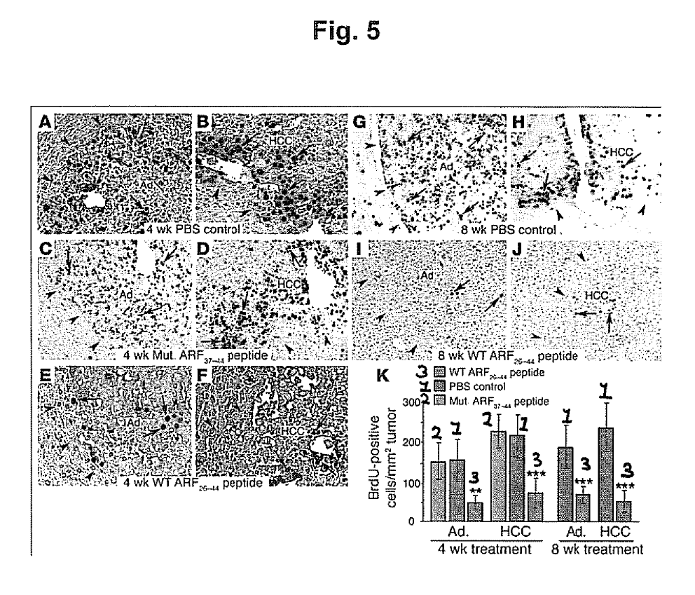

FIGS. 5A through 5K show experimental results demonstrating that the cell penetrating WT ARF 26-44 peptide diminishes proliferation of mouse hepatic tumors in mice treated with the peptide. FIGS. 5A-5J are photomicrographs showing BrdU incorporation detected by immunohistochemical staining of liver tumor sections with monoclonal BrdU antibody from mice treated with the indicated cell penetrating ARF peptides or PBS. FIG. 5K is a graph of mean number of BrdU positive cells per mm² liver tumor (±SD) following treatment with WT ARF 26-44 peptide or Mutant ARF 37-44 peptide or PBS. The asterisks indicate statistically significant changes: **P<0.01 and ***P<0.001. Magnification for A-J is 200×. Ad., hepatic adenoma.

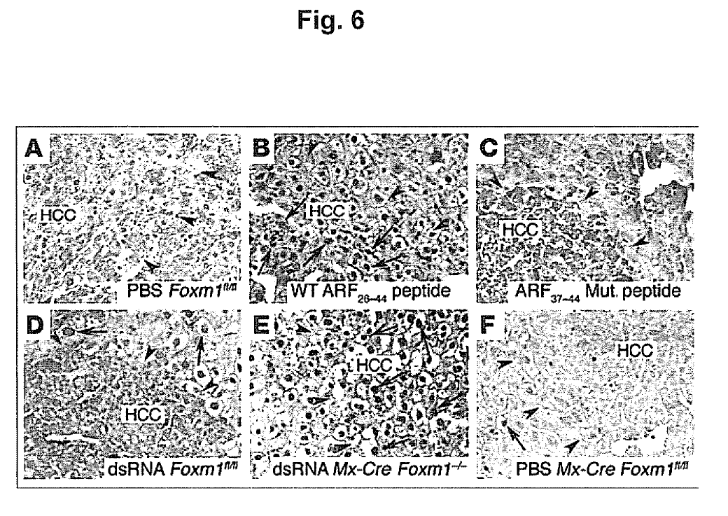

FIGS. 6A through 6F. shows that WT ARF 26-44 peptide treatment causes nuclear accumulation of p27^{Kip1} protein in mouse HCC tumors. FIGS. 6A-6F shows nuclear accumulation of p27^{Kip1} protein in HCC tumors from WT ARF 26-44 peptide treated mice and dsRNA treated Mx-Cre Foxm1 −/− mice. Foxm1 f1/f1 mice were induced for hepatic tumors with DEN/PB treatment and then treated with daily intraperitoneal (IP) injections of 5 mg/Kg body weight of cell penetrating WT (ARG)₉ ARF 26-44 (WT ARF 26-44) peptide (FIG. 6B) or Mutant (ARG)₉ ARF 37-44 (Mut. ARF 37-44). FIGS. 6D-6F are photomicrographs showing the Foxm1 gene genetically deleted in preexisting liver tumors in dsRNA Mx-Cre Foxm1 −/− mice versus control dsRNA Foxm1 f1/f1 and PBS Mx-Cre Foxm1 f1/f1. Liver tumor sections from indicated mice were immunohistochemically stained with the p27^{Kip1} antibody. Arrows depict nuclear staining for p27^{Kip1} protein and arrowheads show liver tumor margins.

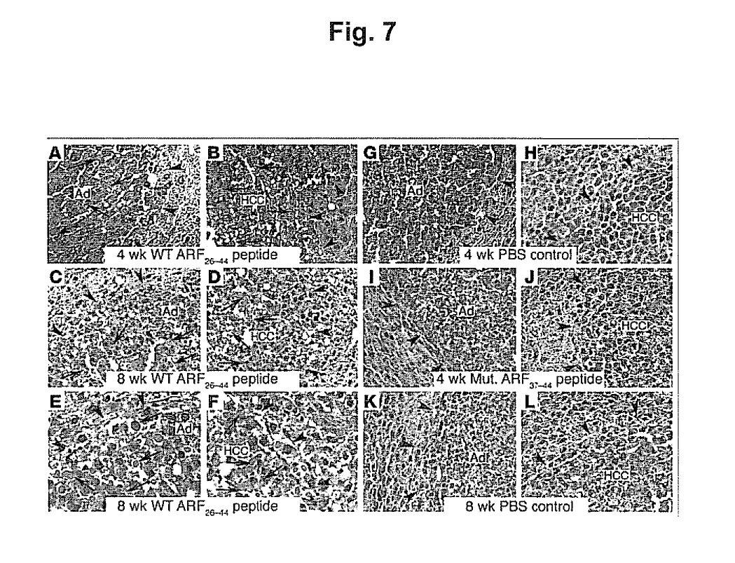

FIGS. 7A through 7L show Hematoxylin and Eosin stained mouse liver tumors from mice treated with WT ARF 26-44 peptide. Foxm1 f1/f1 mice were induced for hepatic tumors with DEN/PB treatment and then treated with daily intraperitoneal (IP) injections at dosages of 5 mg/Kg body weight with cell penetrating WT ARF 26-44 peptide or Mutant ARF 37-44 peptide for 4 or 8 weeks. Arrows depict red-staining cells undergoing apoptosis and arrow heads show liver tumor margins. FIGS. 7A-7F are photomicrographs of Hematoxylin and Eosin (H&E) stained liver tumor sections from WT ARF 26-44 peptide treated mice showing that many of the hepatic adenomas and HCC tumor cells stained red and were rounded up, indicative of apoptosis. FIGS. 7E and 7F are higher magnification photomicrographs of the stained sections shown in FIGS. 7C and 7D. No red staining apoptotic cells were found in either the surrounding, normal liver tissue or in liver tumors from dsRNA (CKO) Foxm1 −/− mice. No red staining tumor cells were found in H&E stained liver tumor sections from mice treated with either PBS or mutant ARF 37-44 peptide (shown in FIGS. 7G-7L).

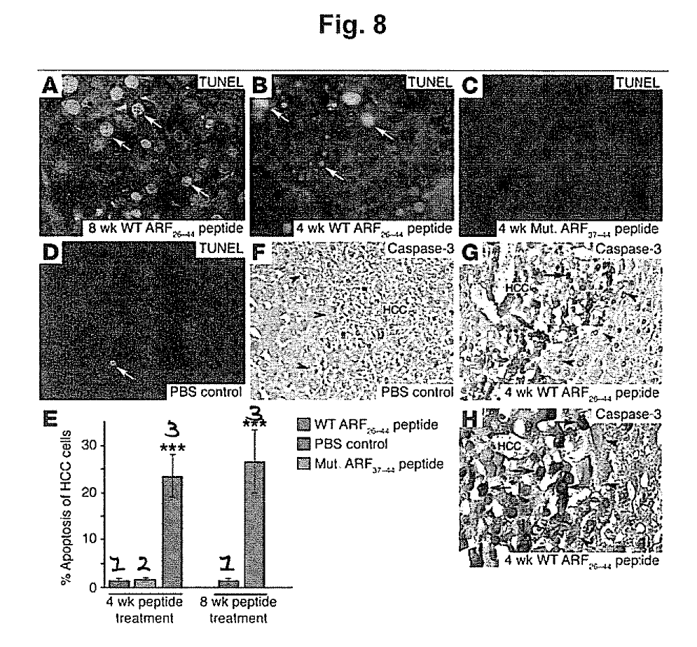

FIGS. 8A through 8H show induction of selective apoptosis in mouse HCC following WT ARF 26-44 peptide treatment. FIGS. 8A-8D are photomicrographs showing liver tumor sections stained for apoptotic cells using the TUNEL assay. FIG. 8E is a graphic quantification of TUNEL positive staining cells. Three asterisks indicate statistically significant change at ***P<0.001. FIGS. 8F-8H shows that selective apoptosis is detected in HCC tumor cells in mice treated with WT ARF 26-44 peptide by immunostaining with antibody specific to proteolytically cleaved activated Caspase 3 protein. Arrows depict nuclear staining for activated Caspase 3 protein and arrowheads show liver tumor margins. Magnification, ×400 (FIGS. 8A-8D and 8H); ×200 (FIGS. 8F and 8G).

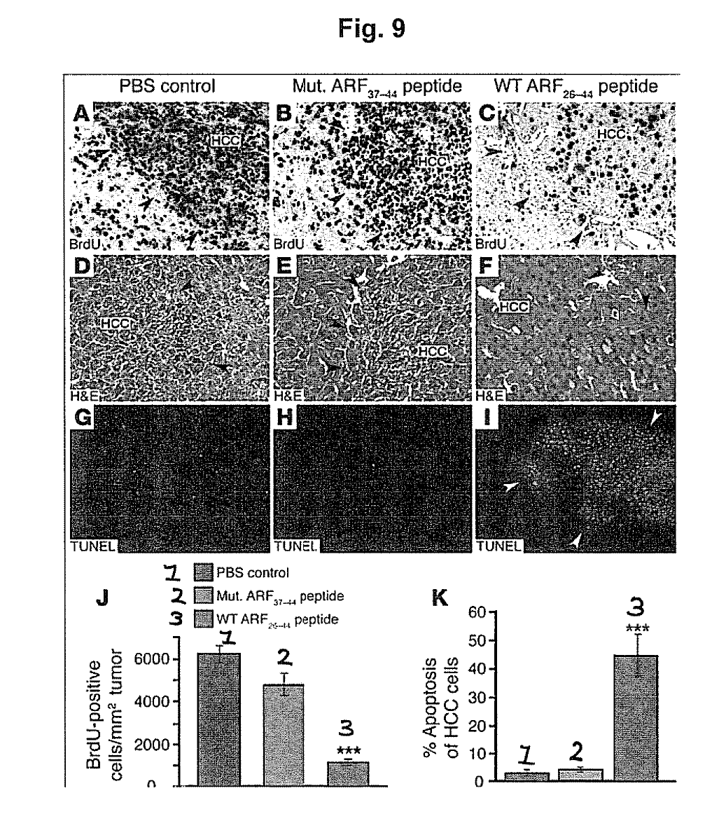

FIGS. 9A through 9K show that WT ARF 26-44 peptide treatment reduced proliferation and increased apoptosis of HCC induced in ARF −/− Rosa26 FoxM1b Transgenic (TG) mice by DEN/PB. Highly proliferative HCC tumors were induced in ARF −/− Rosa26 FoxM1b transgenic (TG) mice following DEN/PB treatment. The ARF −/− Rosa26 FoxM1b transgenic (TG) mice received daily intraperitoneal (IP) injections of the cell penetrating WT ARF 26-44 peptide (inhibitor of FoxM1 function) or Mutant ARF 37-44 peptide or PBS for 4 weeks. FIGS. 9A-9C are photomicrographs showing liver tumor sections subjected to immunohistochemical staining with BrdU monoclonal antibody to determine HCC proliferation. Liver tumor sections were histologically stained with Hematoxylin and Eosin (H&E; FIGS. 9D-9E) to identify red apoptotic cells or stained for apoptosis using the TUNEL assay (FIGS. 9G-9I). FIGS. 9A-9F are 200× magnification and FIGS. 9G-9I are 100× magnification. Black arrowheads indicate the boundaries of the HCC tumor and white arrowheads (FIG. 9I) indicate boundaries of the HCC region. FIG. 9J is a graph depicting the number of BrdU positive cells per mm² liver tumor tissue (±SD). FIG. 9K is a graph depicting the TUNEL-positive cells in HCC representing the percent HCC apoptosis (±SD). P values calculated by Student's t test: ***P<0.001.

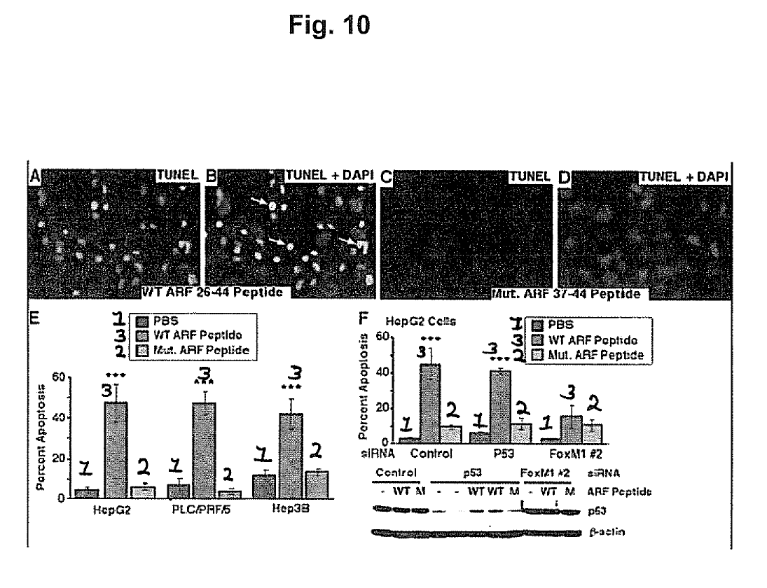

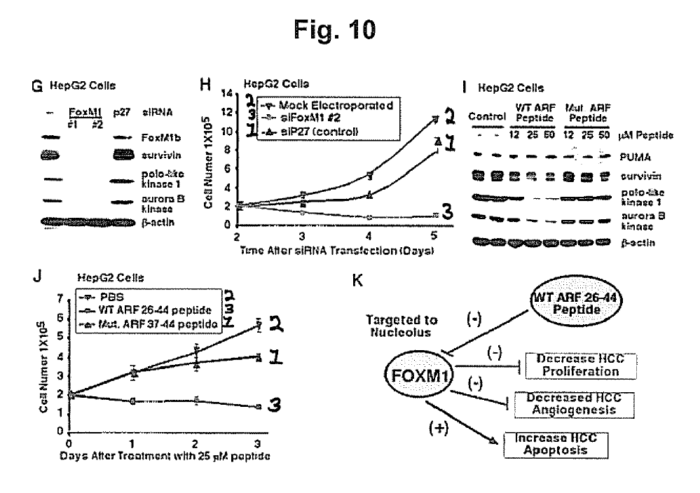

FIGS. 10A through 10K shows WT ARF 26-44 peptide induced apoptosis of Human hepatoma cell lines. Human hepatoma HepG2 (FIGS. 10A-10E), PLC/PRF/5 (express p53 mutant protein) or Hep3B (p53 deficient) cells were treated for 24 hours with 25 μM of cell penetrating WT ARF 26-44 or mutant ARF 37-44 peptide and then analyzed for apoptosis by TUNEL assay and percent apoptosis was calculated ±SD (FIG. 10E; ***P<0.001). Nuclei of HepG2 cells were counterstained with DAPI (FIGS. 10A and 10C) and then merged with TUNEL staining (FIGS. 10B and 10D); TUNEL positive nuclei was indicated by white arrows (FIGS. 10B and 10D). FIG. 10F is a graph of WT ARF 26-44 peptide treated HepG2 cells showing that apoptosis was induced in p53-depleted cells but not in FoxM1-deficient cells. Western blot analysis below the graph shows effective down-regulation of p53 protein levels following p53 siRNA electroporation, and that treatment with WT ARF 26-44 (WT) or mutant ARF 37-44 peptide (M) does not alter p53 protein levels. FIGS. 10G and 10I show Western blot analysis of protein expression of survivin, polo-like kinase 1 (PLK1) and aurora B kinase, in HepG2 cells 48 hours after electroporation with siFoxM1 no. 2 or p27 siRNA duplexes (FIG. 10G), or treatment with WT or mutant ARF peptide (FIG. 10I). FIG. 10J shows a growth curve of HepG2 cells at the indicated days following siRNA transfection (10H) or at the indicated days after ARF peptide treatment (10J). FIG. 10K shows a model summarizing findings with cell penetrating WT ARF 26-44 peptide described in the Examples.

FIG. 11 depicts the amino acid sequence of full length p19ARF protein (SEQ ID NO:7; Quelle et al., 1995, Cell 83: 993-1000)

### DETAILED DESCRIPTION OF THE PREFERRED EMBODIMENTS

The techniques and procedures described herein can be generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Conventional techniques can be used for chemical syntheses, chemical analyses, pharmaceutical preparation, formulation, and delivery, and treatment of patients.

Unless otherwise required by context, singular terms used herein shall include pluralities and plural terms used herein shall include the singular.

The term “isolated protein” referred to herein means a protein encoded by a nucleic acid including, inter alia, genomic DNA, cDNA, recombinant DNA, recombinant RNA, or nucleic acid of synthetic origin or some combination thereof, which (1) is free of at least some proteins with which it would normally be found, (2) is essentially free of other proteins from the same source, e.g., from the same cell or species, (3) is expressed by a cell from a different species, (4) has been separated from at least about 50 percent of polynucleotides, lipids, carbohydrates, or other materials with which it is naturally found when isolated from the source cell, (5) is not linked (by covalent or noncovalent interaction) to all or a portion of a polypeptide to which the “isolated protein” is linked in nature, (6) is operatively linked (by covalent or noncovalent interaction) to a polypeptide with which it is not linked in nature, or (7) does not occur in nature. Preferably, the isolated protein is substantially free from other contaminating proteins or polypeptides or other contaminants that are found in its natural environment that would interfere with its therapeutic, diagnostic, prophylactic or research use.

The phrase “a peptide having an amino acid sequence identified by SEQ ID NO:4” refers to a peptide comprising at least the amino acid sequence as set forth in SEQ SEQ ID NO:4.

The terms “polypeptide” or “protein” is used herein to refer to native proteins, that is, proteins produced by naturally-occurring and specifically non-recombinant cells, or by genetically-engineered or recombinant cells, and comprise molecules having the amino acid sequence of the native protein, or sequences that have deletions, additions, and/or substitutions of one or more amino acids of the native sequence. In addition, the terms “polypeptide” and “protein” as used herein specifically encompass peptides that can inhibit FoxM1B activity, including the (D-Arg)₉-p19ARF 26-44 peptide (SEQ ID NO: 3; rrrrrrrrrKFVRSRRPRTASCALAFVN), the p19^{ARF} 26-44 peptide (SEQ ID NO: 4; KFVRSRRPRTASCALAFVN), and the p19^{ARF} 26-55 peptide (SEQ ID NO: 5; KFVRSRRPRTASCALAFVNMLLRLERILRR), or species thereof that have deletions, additions, and/or substitutions of one or more amino acids of SEQ ID NO: 3, SEQ ID NO: 4, or SEQ ID NO: 5 having the ability to inhibit FoxM1B activity. Assays for determining if such species can inhibit FoxM1B activity are described, for example, in U.S. patent application Ser. No. 10/809,144 filed Mar. 25, 2004, incorporated herein by reference in its entirety.

The term “naturally-occurring” as used herein refers to an object that can be found in nature, for example, a polypeptide or polynucleotide sequence that is present in an organism (including a virus) that can be isolated from a source in nature and which has not been intentionally modified by man. The term “naturally occurring” or “native” when used in connection with biological materials such as nucleic acid molecules, polypeptides, host cells, and the like, refers to materials which are found in nature and are not manipulated by man. Similarly, “recombinant,” “non-naturally occurring” or “non-native” as used herein refers to a material that is not found in nature or that has been structurally modified or synthesized by man.

The term “fragment” as used herein refers to a portion less than the whole. For example, a DNA fragment refers to a DNA molecule containing a polynucleotide sequence that is less than the full length DNA; a protein fragment refers to a protein, a polypeptide, or a peptide that is less than the full length protein; and a fragment of a peptide refers to a peptide shorter than the full length peptide.

As used herein, the twenty conventional amino acids and their abbreviations follow conventional usage. See IMMUNOLOGY—A SYNTHESIS, 2nd Edition, (E. S. Golub and D. R. Gren, Eds.), 1991, Sinauer Associates, Sunderland, Mass., which is incorporated herein by reference for any purpose. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts or comprising functional domains). In certain embodiments, a conservative amino acid substitution does not substantially change the structural characteristics of the parent sequence (e.g., a replacement amino acid should not disrupt secondary structure that characterizes the parent or native protein, such as a helix). Examples of art-recognized polypeptide secondary and tertiary structures are described in PROTEINS, STRUCTURES AND MOLECULAR PRINCIPLES (Creighton, Ed.), 1984, W. H. New York: Freeman and Company; INTRODUCTION TO PROTEIN STRUCTURE (Branden and Tooze, eds.), 1991, New York: Garland Publishing; and Thornton et at., 1991, Nature 354: 105, which are each incorporated herein by reference.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

In contrast, non-conservative substitutions may involve the exchange of a member of one of these classes for a member from another class. Such substituted residues may be introduced into regions of a protein or polypeptide that are homologous with non-human orthologs thereof, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte et al., 1982, J. Mol. Biol. 157:105-131).

The importance of the hydropathic amino acid index in conferring interactive biological function on a protein is understood in the art (see, for example, Kyte et al., 1982, ibid.). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen-binding or immunogenicity, i.e., with a biological property of the protein.

As described in U.S. Pat. No. 4,554,101, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Exemplary amino acid substitutions are set forth in Table 1.

TABLE 1
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, Gln, Asn, | Arg |
| | 1,4 Diamine-butyric Acid | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan can determine suitable variants of the polypeptide as set forth herein using well-known techniques. In certain embodiments, one skilled in the art can identify suitable areas of the molecule that can be changed without destroying activity by targeting regions not understood to be important for activity. In certain embodiments, residues and portions of the molecules can be identified that are conserved among similar polypeptides. In certain embodiments, even areas that are important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled worker can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze three-dimensional structure and amino acid sequence in relation to that structure in similar polypeptides. In view of such information, one skilled in the art can predict the alignment of amino acid residues of a polypeptide with respect to its three dimensional structure. In certain embodiments, one skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened for the ability to inhibit FoxM1B activity using assays described, for example, in U.S. patent application Ser. No. 10/809,144 filed Mar. 25, 2004. Such variants can be used to gather information about suitable variants. For example, if it was discovered that a change to a particular amino acid residue resulted in destroyed, undesirably reduced, or produced an unsuitable activity, variants with such a change can be avoided. In other words, based on information gathered from such routine experiments, one skilled in the art can readily determine the amino acids where further substitutions should be avoided either alone or in combination with other mutations.

Stereoisomers (e.g., D-amino acids) of the twenty conventional amino acids, non-naturally occurring amino acids such as α-,α-disubstituted amino acids, N-alkyl amino acids, lactic acid, and other unconventional amino acids may also be suitable components for polypeptides of the present invention. Examples of unconventional amino acids include but are not limited to: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Also provided are related compounds within the understanding of those with skill in the art, such as chemical mimetics, organomimetics or peptidomimetics. As used herein, the terms “mimetic,” “peptide mimetic,” “peptidomimetic,” “organomimetic” and “chemical mimetic” are intended to encompass peptide derivatives, peptide analogues and chemical compounds having an arrangement of atoms is a three-dimensional orientation that is equivalent to that of a peptide of the invention. It will be understood that the phrase “equivalent to” as used herein is intended to encompass compounds having substitution of certain atoms or chemical moieties in said peptide with moieties having bond lengths, bond angles and arrangements thereof in the mimetic compound that produce the same or sufficiently similar arrangement or orientation of said atoms and moieties to have the biological function of the peptides of the invention. In the peptide mimetics of the invention, the three-dimensional arrangement of the chemical constituents is structurally and/or functionally equivalent to the three-dimensional arrangement of the peptide backbone and component amino acid sidechains in the peptide, resulting in such peptido-, organo- and chemical mimetics of the peptides of the invention having substantial biological activity. These terms are used according to the understanding in the art, as illustrated for example by Fauchere, 1986, Adv. Drug Res. 15: 29; Veber & Freidinger, 1985, TINS p. 392; and Evans et al., 1987, J. Med. Chem. 30: 1229, incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo- and chemical mimetics can be designed to fit each pharmacophore with current computer modeling software (computer aided drug design). Said mimetics are produced by structure-function analysis, based on the positional information from the substituent atoms in the peptides of the invention.

Peptides as provided by the invention can be advantageously synthesized by any of the chemical synthesis techniques known in the art, particularly solid-phase synthesis techniques, for example, using commercially-available automated peptide synthesizers. The mimetics of the present invention can be synthesized by solid phase or solution phase methods conventionally used for the synthesis of peptides (see, for example, Merrifield, 1963, J. Amer. Chem. Soc. 85: 2149-54; Carpino, 1973, Acc. Chem. Res. 6: 191-98; Birr, 1978, ASPECTS OF THE MERRIFIELD PEPTIDE SYNTHESIS, Springer-Verlag: Heidelberg; THE PEPTIDES: ANALYSIS, SYNTHESIS, BIOLOGY, Vols. 1, 2, 3, 5, (Gross & Meinhofer, eds.), Academic Press: New York, 1979; Stewart et al., 1984, SOLID PHASE PEPTIDE SYNTHESIS, 2nd. ed., Pierce Chem. Co.: Rockford, Ill.; Kent, 1988, Ann. Rev. Biochem. 57: 957-89; and Gregg et al., 1990, Int. J. Peptide Protein Res. 55: 161-214, which are incorporated herein by reference in their entirety.)

The use of solid phase methodology is preferred. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods or the like. Cycles of deprotection, neutralization and coupling of successive protected amino acid derivatives are used to link the amino acids from the C-terminus according to the amino acid sequence. For some synthetic peptides, an FMOC strategy using an acid-sensitive resin may be used. Preferred solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A particularly preferred protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues can be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-1-yl), 1,1,3,3-tetramethyluronium hexafluoro-phosphate) in the presence of HOBt or HOAt (7-azahydroxybenztriazole) is preferred.

The solid phase method can be carried out manually, although automated synthesis on a commercially available peptide synthesizer (e.g., Applied Biosystems 431A or the like; Applied Biosystems, Foster City, Calif.) is preferred. In a typical synthesis, the first (C-terminal) amino acid is loaded on the chlorotrityl resin. Successive deprotection (with 20% piperidine/NMP(N-methylpyrrolidone)) and coupling cycles according to ABI FastMoc protocols (ABI user bulletins 32 and 33, Applied Biosystems are used to build the whole peptide sequence. Double and triple coupling, with capping by acetic anhydride, may also be used.

The synthetic mimetic peptide is cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, can be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization out of the appropriate base.

Generally, those skilled in the art will recognize that peptides as described herein may be modified by a variety of chemical techniques to produce compounds having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric and other organic salts, or may be converted to an amide. Thiols can be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogues with the same or similar desired biological activity as the corresponding peptide compound but with more favorable activity than the peptide with respect to solubility, stability, and susceptibility to hydrolysis and proteolysis. Such derivatives and analogues include peptides modified at the N-terminal amino group, the C-terminal carboxyl group, and/or changing one or more of the amido linkages in the peptide to a non-amido linkage. It will be understood that two or more such modifications can be coupled in one peptide mimetic structure (e.g., modification at the C-terminal carboxyl group and inclusion of a —CH₂— carbamate linkage between two amino acids in the peptide).

Amino terminus modifications include alkylating, acetylating, adding a carbobenzoyl group, and forming a succinimide group. Specifically, the N-terminal amino group can then be reacted to form an amide group of the formula RC(O)NH— where R is alkyl, preferably lower alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—. Alternatively, the amino terminus can be covalently linked to succinimide group by reaction with succinic anhydride. An approximately equimolar amount or an excess of succinic anhydride (e.g., about 5 equivalents) are used and the terminal amino group is converted to the succinimide by methods well known in the art including the use of an excess (e.g., ten equivalents) of a tertiary amine such as diisopropylethylamine in a suitable inert solvent (e.g., dichloromethane), as described in Wollenberg et al., U.S. Pat. No. 4,612,132, is incorporated herein by reference in its entirety. It will also be understood that the succinic group can be substituted with, for example, C₂- through C₆-alkyl or —SR substituents, which are prepared in a conventional manner to provide for substituted succinimide at the N-terminus of the peptide. Such alkyl substituents are prepared by reaction of a lower olefin (C₂- through C₆-alkyl) with maleic anhydride in the manner described by Wollenberg et al., supra., and —SR substituents are prepared by reaction of RSH with maleic anhydride where R is as defined above. In another advantageous embodiments, the amino terminus is derivatized to form a benzyloxycarbonyl-NH— or a substituted benzyloxycarbonyl-NH— group. This derivative is produced by reaction with approximately an equivalent amount or an excess of benzyloxycarbonyl chloride (CBZ—Cl) or a substituted CBZ—Cl in a suitable inert diluent (e.g., dichloromethane) preferably containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl and preferably lower alkyl. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes). Carbamate groups are produced at the amino terminus by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate, where R is alkyl, preferably lower alkyl. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups are formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (e.g., —C(O)OR where R is alkyl and preferably lower alkyl), resins used to prepare the peptide acids are employed, and the side chain protected peptide is cleaved with base and the appropriate alcohol, e.g., methanol. Side chain protecting groups are then removed in the usual fashion by treatment with hydrogen fluoride to obtain the desired ester. In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain Protected peptide from the support yields the free peptide amide and reaction with an alkylamine or a dialkylamine yields a side chain protected alkylamide or dialkylamide (i.e., the C-terminus is —C(O)NRR₁, where R and R₁ are alkyl and preferably lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by displacement of the —OH or the ester (—OR) of the carboxyl group or ester respectively with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH₂Cl₂), dimethyl formamide (DMF), or mixtures thereof. The cyclic peptide is then formed by displacement of the activated ester with the N-terminal amine. Cyclization, rather than polymerization, can be enhanced by use of very dilute solutions according to methods well known in the art.

Peptide mimetics as understood in the art and provided by the invention are structurally similar to the paradigm peptide of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, 1983, in CHEMISTRY AND BIOCHEMISTRY OF AMINO ACIDS, PEPTIDES, AND PROTEINS, (Weinstein, ed.), Marcel Dekker: New York, p. 267; Spatola, 1983, Peptide Backbone Modifications 1: 3; Morley, 1980, Trends Pharm. Sci. pp. 463-468; Hudson et al., 1979, Int. J. Pept. Prot. Res. 14: 177-185; Spatola et al., 1986, Life Sci. 38: 1243-1249; Hann, 1982, J. Chem. Soc. Perkin Trans. 1307-314; Almquist et al., 1980, J. Med. Chem. 23: 1392-1398; Jennings-White et al., 1982, Tetrahedron Lett. 23: 2533; Szelke et al., 1982, European Patent Application, Publication No. EP045665A; Holladay et al., 1983, Tetrahedron Lett. 24: 4401-4404; and Hruby, 1982, Life Sci. 31: 189-199, each of which is incorporated herein by reference. Such peptide mimetics may have significant advantages over polypeptide embodiments, including, for example: being more economical to produce, having greater chemical stability or enhanced pharmacological properties (such half-life, absorption, potency, efficacy, etc.), reduced antigenicity, and other properties.

Mimetic analogs of the tumor-inhibiting peptides of the invention may also be obtained using the principles of conventional or rational drug design (see, Andrews et al., 1990, Proc. Alfred Benzon Symp. 28: 145-165; McPherson, 1990, Eur. J. Biochem. 189: 1-24; Hol et al., 1989a, in MOLECULAR RECOGNITION: CHEMICAL AND BIOCHEMICAL PROBLEMS, (Roberts, ed.); Royal Society of Chemistry; pp. 84-93; Hol, 1989b, Arzneim-Forsch. 39:1016-1018; Hol, 1986, Agnew Chem. Int. Ed. Engl. 25: 767-778, the disclosures of which are herein incorporated by reference).

In accordance with the methods of conventional drug design, the desired mimetic molecules are obtained by randomly testing molecules whose structures have an attribute in common with the structure of a “native” peptide. The quantitative contribution that results from a change in a particular group of a binding molecule can be determined by measuring the biological activity of the putative mimetic in comparison with the tumor-inhibiting activity of the peptide. In a preferred embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the tumor-inhibiting peptides of the invention, as disclosed herein.

The preferred method for performing rational mimetic design employs a computer system capable of forming a representation of the three-dimensional structure of the peptide, such as those exemplified by Hol, 1989a, ibid.; Hol, 1989b, ibid.; and Hol, 1986, ibid. Molecular structures of the peptido-, organo- and chemical mimetics of the peptides of the invention are produced according to those with skill in the art using computer-assisted design programs commercially available in the art. Examples of such programs include SYBYL 6.5®, HQSAR™, and ALCHEMY 2000™(Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo- and chemical mimetics produced using the peptides disclosed herein using, for example, art-recognized molecular modeling programs are produced using conventional chemical synthetic techniques, most preferably designed to accommodate high throughput screening, including combinatorial chemistry methods. Combinatorial methods useful in the production of the peptido-, organo- and chemical mimetics of the invention include phage display arrays, solid-phase synthesis and combinatorial chemistry arrays, as provided, for example, by SIDDCO, Tuscon, Ariz.; Tripos, Inc.; Calbiochem/Novabiochem, San Diego, Calif.; Symyx Technologies, Inc., Santa Clara, Calif.; Medichem Research, Inc., Lemont, Ill.; Pharm-Eco Laboratories, Inc., Bethlehem, Pa.; or N.V. Organon, Oss, Netherlands. Combinatorial chemistry production of the peptido-, organo- and chemical mimetics of the invention are produced according to methods known in the art, including but not limited to techniques disclosed in Terrett, 1998, COMBINATORIAL CHEMISTRY, Oxford University Press, London; Gallop et al., 1994, “Applications of combinatorial technologies to drug discovery. 1. Background and peptide combinatorial libraries,” J. Med. Chem. 37: 1233-51; Gordon et al., 1994, “Applications of combinatorial technologies to drug discovery. 2. Combinatorial organic synthesis, library screening strategies, and future directions,” J. Med. Chem. 37: 1385-1401; Look et al., 1996, Bioorg. Med. Chem. Lett. 6: 707-12; Ruhland et al., 1996, J. Amer. Chem. Soc. 118: 253-4; Gordon et al., 1996, Acc. Chem. Res. 29: 144-54; Thompson & Ellman, 1996, Chem. Rev. 96: 555-600; Fruchtel & Jung, 1996, Angew. Chem. Int. Ed. Engl. 35: 17-42; Pavia, 1995, “The Chemical Generation of Molecular Diversity”, Network Science Center, www.netsci.org; Adnan et al., 1995, “Solid Support Combinatorial Chemistry in Lead Discovery and SAR Optimization,” Id., Davies and Briant, 1995, “Combinatorial Chemistry Library Design using Pharmacophore Diversity,” Id., Pavia, 1996, “Chemically Generated Screening Libraries: Present and Future,” Id.; and U.S. Pat. No. 5,880,972 to Horlbeck; U.S. Pat. No. 5,463,564 to Agrafiotis et al.; U.S. Pat. No. 5,331,573 to Balaji et al.; and U.S. Pat. No. 5,573,905 to Lerner et al.

A peptide of the invention can be produced using various methods that are established in the art, including chemical synthesis or recombinant methods. Recombinant DNA techniques are well known in the art. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Methods of chemical synthesis of peptides typically involve solid-state approaches, but can also utilize solution-based chemistries or combinations of solid-state and solution approaches. Examples of solid-state methodologies for synthesizing proteins are described by Merrifield, 1964, J. Am. Chem. Soc. 85:2149; and Houghton, 1985, Proc. Natl. Acad. Sci. 82:5132. Fragments of a peptide of the invention can also be synthesized and then joined together. Methods for conducting such reactions are described by Grant, 1992, Synthetic Peptides: A User Guide, W.H. Freeman and Co., N.Y.; and in “Principles of Peptide Synthesis,” 1993 (Bodansky and Trost, ed.), Springer-Verlag, Inc. N.Y. Further guidance on methods for preparing peptides sufficient to guide the skilled practitioner in the preparation of the peptides of the invention as described herein is provided by: Liu et al., 1996, J. Am. Chem. Soc. 118:307-312; Kullmann, 1987, Enzymatic Peptide Synthesis, CRC Press, Boca Raton, Fla., pp. 41-59; Dryland et al., 1986, J. Chem. Soc., Perkin Trans. 1:125-137; Jones, 1991, The Chemical Synthesis of Peptides, Clarendon Press; and Bodanszky, M. and Bodanszky A., 1994, The Practice of Peptide Synthesis, 2^{nd} Ed., Springer-Verlag).

In certain embodiments, a peptide of the invention can be pegylated. As used herein, the terms “pegylated” and “pegylation” refers generally to the process of chemically modifying a peptide of the invention by covalent attachment of one or more molecules of polyethylene glycol or a derivative thereof, such as by reacting a polyalkylene glycol, preferably an activated polyalkylene glycol, with a facilitator such as an amino acid, e.g. lysine, to form a covalent bond. Although “pegylation” is often carried out using polyethylene glycol or derivatives thereof, such as methoxy polyethylene glycol, the term as used herein also includes any other useful polyalkylene glycol, such as, for example polypropylene glycol. As used herein, the term “PEG” refers to polyethylene glycol and its derivatives as understood in the art (see for example U.S. Pat. Nos. 5,445,090, 5,900,461, 5,932,462, 6,436,386, 6,448,369, 6,437,025, 6,448,369, 6,495,659, 6,515,100, and 6,514,491). A variety of strategies can be used for pegylation of a peptide of the invention (see, e.g., Veronese, 2001, Biomaterials 22:405-417; Roberts et al., 2002, Advanced Drug Delivery Reviews 54:459-476; Delgado et al., Crit. Rev. Thera. Drug Carrier Sys. 9:249-304, 1992; Francis et al., 1998, Intern. J. of Hematol. 68:1-18; U.S. Pat. No. 4,002,531; U.S. Pat. No. 5,349,052; WO 95/06058; WO 98/32466; U.S. Pat. No. 4,343,898).

Peptides of the invention can also be modified with a water-soluble polymer other than PEG. Suitable water-soluble polymers or mixtures thereof include, but are not limited to, N-linked or O-linked carbohydrates, sugars (e.g. various polysaccharides such as chitosan, xanthan gum, cellulose and its derivatives, acacia gum, karaya gum, guar gum, carrageenan, and agarose), phosphates, dextran (such as low molecular weight dextran of, for example, about 6 kD), cellulose, or other carbohydrate based polymers.

The Applicants has discovered that the peptide containing amino acid residues 26-44 of p19ARF protein is sufficient in inhibiting FoxM1B activity (See U.S. patent application Ser. No. 10/809,144, incorporated herein by reference in its entirety). The Applicants also discovered that a cell-penetrating molecule, such as a peptide of nine arginine residues (SEQ ID NO:10), covalently linked to the p19ARF26-44 peptide facilitates cell penetration and further enhances the inhibitory effect of the peptide on FoxM1B activity and angiogenesis. It is understood that one of skill in the art would be able to modify the invention by covalently linking other cell-penetrating molecules to a peptide having the sequence identified by SEQ ID NO:4. It is known in the art that protein transduction domains (PTDs) are a group of peptides that can cross biological membranes in a receptor-independent manner. Such non-limiting examples include a PTD with the sequence of 11 amino acid residues YGRKKRRQRRR (SEQ ID NO:8) and variations thereof. For example, one such variation YARAAARQARA (SEQ ID NO:9) has been shown to exhibit good cell-penetrating ability. (Ho et al., Cancer Research 61, 474-477, Jan. 15, 2001) The use of such non-limiting examples of cell-penetrating molecules in conjunction with the claimed peptide is within the scope of the invention.

In certain embodiments, the invention provides methods for inhibiting angiogenesis in a patient comprising administering to the patient, which has at least one tumor cell present in the patient's body, a therapeutically effective amount of a peptide, such as a peptide having an amino acid sequence as set forth in SEQ ID NO: 3 or SEQ ID NO: 4 for a therapeutically effective period of time.

In another embodiment, the invention provides methods for inhibiting angiogenesis in a patient, which does not have tumor cells present in the body, comprising administering to the patient a therapeutically effective amount of a peptide, such as a peptide having an amino acid sequence as set forth in SEQ ID NO: 3 or SEQ ID NO: 4 for a therapeutically effective period of time.

In another embodiment, the invention provides methods for inhibiting tumor growth in an animal comprising by administering to the animal, which has at least one tumor cell present in its body, a therapeutically effective amount of a peptide, such as a peptide having an amino acid sequence as set forth in SEQ ID NO: 3 or SEQ ID NO: 4, or a composition comprising a peptide, such as a peptide having an amino acid sequence as set forth in SEQ ID NO: 3 or SEQ ID NO: 4.

In certain embodiments, the invention provides methods for inhibiting angiogenesis. In a particular embodiment, the methods of the invention comprise administering a peptide, such as a peptide having an amino acid sequence as set forth in SEQ ID NO: 3 or SEQ ID NO: 4, or a composition comprising a peptide, such as a peptide having an amino acid sequence as set forth in SEQ ID NO: 3 or SEQ ID NO: 4, to an animal in need thereof.

As used herein, the term “angiogenesis” refers to the formation of new blood vessels from pre-existing capillaries or post-capillary venules, and includes de novo formation of vessels, for example vessels arising from vasculogenesis, as well as those arising from branching and sprouting of existing vessels, capillaries, and venules. As used herein, the term “vasculogenesis” refers to the formation of new blood vessels arising from angioblasts.

As used herein, the phrase “inhibiting angiogenesis” includes vasculogenesis, and refers to causing a decrease in the extent, amount, or rate of neovascularization, for example by decreasing the extent, amount, or rate of endothelial cell proliferation or migration in a tissue.

The methods of the invention can inhibit a biological process comprising angiogenesis such as angiogenic factor production, angiogenic factor release, endothelial cell receptor binding, endothelial cell activation, endothelial cell migration, proliferation, extracellular matrix (ECM) remodeling, tube formation, vascular stabilization, formation of new blood vessels from existing ones, and consequently the inhibition of angiogenesis-related or dependent diseases.

As used herein, the term “angiogenesis-related disease” or “angiogenesis-dependent disease” includes a disease where the angiogenesis or vasculogenesis sustains or augments a pathological condition. Non-limiting examples of angiogenesis-dependent diseases include inflammatory disorders, such as immune and non-immune inflammation, rheumatoid arthritis, chronic articular rheumatism and psoriasis; disorders associated with inappropriate invasion of vessels, such as diabetic retinopathy, neovascular glaucoma, retinopathy of prematurity, macular degeneration, loss of vision as a result of blood and other retinal fluids leak into the retina, corneal graft rejection, retrolental fibroplasia, rubeosis, capillary proliferation in atherosclerotic plaques and osteoporosis; and cancer, including for example, solid tumors, tumor metastases, liver tumor, prostate cancer, lung cancer, blood born tumors such as leukemias, angiofibromas, Kaposi sarcoma, benign tumors, such as hemangiomas, acoustic neuromas, neurofibromas, trachomas, and pyogenic granulomas, as well as other cancers that require neovascularization to support tumor growth. Additional non-limiting examples of angiogenesis-related or -dependent diseases include, for example, Osler-Webber Syndrome; myocardial angiogenesis; plaque neovascularization; telangiectasia; edema; hemophiliac joints; and wound granulation.

In certain embodiments, the invention provides pharmaceutical compositions comprising a therapeutically effective amount of a peptide, such as a peptide having an amino acid sequence as set forth in SEQ ID NO: 3 or SEQ ID NO: 4, together with a pharmaceutically acceptable diluent, carrier, solubilizer, emulsifier, preservative and/or adjuvant. In other embodiments, the invention provides pharmaceutical compositions that comprise a therapeutically effective amount of a peptide, such as a peptide having an amino acid sequence as set forth in SEQ ID NO: 3 or SEQ ID NO: 4 together with a pharmaceutically acceptable diluent, carrier, solubilizer, emulsifier, preservative and/or adjuvant. Such compounds can be identified in screening methods of the invention.

The term “agent” is used herein to denote a chemical compound, a mixture of chemical compounds, a biological macromolecule, or an extract made from biological materials.

The term “pharmaceutical composition” as used herein refers to a composition comprising a pharmaceutically acceptable carrier, excipient, or diluent and a chemical compound, peptide, or composition as described herein that is capable of inducing a desired therapeutic effect when properly administered to a patient.

The term “therapeutically effective amount” refers to the amount of a peptide, such as a peptide having an amino acid sequence as set forth in SEQ ID NO: 3 or SEQ ID NO: 4, or a composition comprising a peptide, such as a peptide having an amino acid sequence as set forth in SEQ ID NO: 3 or SEQ ID NO: 4, determined to produce a therapeutic response in a mammal. Such therapeutically effective amounts are readily ascertained by one of ordinary skill in the art and using methods as described herein.

As used herein, “substantially pure” means an object species that is the predominant species present (i.e., on a molar basis it is more abundant than any other individual species in the composition). In certain embodiments, a substantially purified fraction is a composition wherein the object species comprises at least about 50 percent (on a molar basis or on a weight or number basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (wherein contaminating species cannot be detected in the composition by conventional detection methods) wherein the composition consists essentially of a single macromolecular species.

The term “patient” includes human and animal subjects.

As used herein, the terms “tumor growth” and “tumor cell proliferation” are used to refer to the growth of a tumor cell. The term “tumor cell” as used herein refers to a cell that is neoplastic. A tumor cell can be benign, i.e. one that does not form metastases and does not invade and destroy adjacent normal tissue, or malignant, i.e. one that invades surrounding tissues, is capable of producing metastases, may recur after attempted removal, and is likely to cause death of the host. Preferably a tumor cell that is subjected to a method of the invention is an epithelial-derived tumor cell, such as a tumor cell derived from skin cells, lung cells, intestinal epithelial cells, colon epithelial cells, testes cells, breast cells, prostate cells, brain cells, bone marrow cells, blood lymphocytes, ovary cells or thymus cells. In one embodiment of the invention, the tumor is a solid tumor. In another embodiment, the tumor has metastasized or will likely metastasize in the patient.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company.

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefor. Pharmaceutical compositions of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the FoxM1B-inhibiting product may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical compositions of the invention can be delivered parenterally. When parenteral administration is contemplated, the therapeutic compositions for use in this invention may be in the form of a pyrogen-free, parenterally acceptable aqueous solution comprising the desired compound identified in a screening method of the invention in a pharmaceutically acceptable vehicle. A particularly suitable vehicle for parenteral injection is sterile distilled water in which the compound identified in a screening method of the invention is formulated as a sterile, isotonic solution, appropriately preserved. Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which may then be delivered via a depot injection. Formulation with hyaluronic acid has the effect of promoting sustained duration in the circulation. Implantable drug delivery devices may be used to introduce the desired molecule. Any other parenteral delivery means is contemplated for use in conjunction of the current invention.

The compositions may be formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

The pharmaceutical compositions of the invention can be delivered through the digestive tract, such as orally. The preparation of such pharmaceutically acceptable compositions is within the skill of the art. Compositions of the invention that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of a peptide, such as a peptide having an amino acid sequence as set forth in SEQ ID NO: 3 or SEQ ID NO: 4. Diluents, flavorings, low melting point waxes, vegetable oils, lubricants, suspending agents, tablet disintegrating agents, and binders may also be employed.

A pharmaceutical composition may involve an effective quantity of a peptide, such as a peptide having an amino acid sequence as set forth in SEQ ID NO: 3 or SEQ ID NO: 4 in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are evident to those skilled in the art, including formulations involving a peptide, such as a peptide having an amino acid sequence as set forth in SEQ ID NO: 3 or SEQ ID NO: 4 in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT Application No. PCT/US93/00829, which describes the controlled release of porous polymeric microparticles for the delivery of pharmaceutical compositions. Sustained-release preparations may include semipermeable polymer matrices in the form of shaped articles, e.g. films, or microcapsules, polyesters, hydrogels, polylactides (U.S. Pat. No. 3,773,919 and EP 058,481), copolymers of L-glutamic acid and gamma ethyl-L-glutamate (Sidman et al., 1983, Biopolymers 22: 547-556), poly (2-hydroxyethyl-methacrylate) (Langer et al., 1981, J. Biomed. Mater. Res. 15: 167-277) and Langer, 1982, Chem. Tech. 12: 98-105), ethylene vinyl acetate (Langer et al., id.) or poly-D(−)-3-hydroxybutyric acid (EP 133,988). Sustained release compositions may also include liposomes, which can be prepared by any of several methods known in the art. See e.g., Eppstein et al., 1985, Proc. Natl. Acad. Sci. USA 82: 3688-3692; EP 036,676; EP 088,046 and EP 143,949.

The pharmaceutical composition to be used for in vivo administration typically is sterile and pyrogen-free. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The present invention is directed to kits for producing a single-dose administration unit. Kits according to the invention may each contain both a first container having a dried protein compound identified in a screening method of the invention and a second container having an aqueous formulation, including for example single and multi-chambered pre-filled syringes (e.g., liquid syringes, lyosyringes or needle-free syringes).

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg. In other embodiments, the dosage may range from 0.1 mg/kg to 10 mg/kg body weight. In yet other embodiments, the patient is subjected to 0.1, 1, 5, or 10 mg/kg body weight of the peptide.

The dosing frequency will depend upon the pharmacokinetic parameters of a peptide, such as a peptide having an amino acid sequence as set forth in SEQ ID NO: 3 or SEQ ID NO: 4 in the formulation. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, subcutaneous, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In certain embodiments, it may be desirable to use a peptide, such as a peptide having an amino acid sequence as set forth in SEQ ID NO: 3 or SEQ ID NO: 4 in an ex vivo manner. In such instances, cells, tissues or organs that have been removed from the patient are exposed to pharmaceutical compositions of the invention or a recombinant nucleic acid construct encoding a peptide, such as a peptide having an amino acid sequence as set forth in SEQ ID NO: 3 or SEQ ID NO: 4 after which the cells, tissues and/or organs are subsequently implanted back into the patient.

Pharmaceutical compositions of the invention can be administered alone or in combination with other therapeutic agents, in particular, in combination with other cancer therapy agents. Such agents generally include radiation therapy or chemotherapy. Chemotherapy, for example, can involve treatment with one or more of the following agents: anthracyclines, taxol, tamoxifene, doxorubicin, 5-fluorouracil, and other drugs known to one skilled in the art. In patient with non-cancer angiogenesis-dependent diseases, pharmaceutical compositions of the invention can be administered alone or in combination with other therapeutic agents, for example, agents for treating inflammatory disorders such as rheumatoid arthritis or psoriasis, and agents for treating disorders associated with inappropriate invasion of vessels.

In one embodiment, the methods of the invention can be advantageously performed after surgery where solid tumors have been removed as a prophylaxis against metastases.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### (D-ARG)₉-ARF 26-44 Peptide Inhibits Angiogenesis

C57BL/6 mice containing Foxm1 LoxP/LoxP (f1/f1) targeted allege generated as described in Wang et al. (2002, Proc. Natl. Acad. Sci. USA 99:16881-16886) were bred into the C57BL/6 mouse background for 8 generations. Type I interferon inducible Mx promoter driven Cre Recombinase (Mx-Cre) transgenic mice (TG) C57BL/6 mice (C57BL/6-TgN Mx-Cre) were purchased from The Jackson Laboratory (Bar Harbor, Me.). Mx-Cre TG C57BL/6 mice were bred with Foxm1 f1/f1 C57BL/6 mice and the offspring were screened for Mx-Cre Foxm1 f1/+ mice. The mice were then backcrossed with Foxm1 f1/f1/C57BL/6 mice to generate Mx-Cre Foxm1 f1/f1 C57BL/6 mice.

At 14 days after birth, the Mx-Cre Foxm1 f1/f1 C57BL/6 mice were injected intraperitoneally (IP) with the tumor initiator diethylnitrosamine (5 μg of Diethylnitrosamine (DEN)/g body weight; Sigma-Aldrich, St. Louis, Mo.) to induce liver tumors. Two weeks later, male mice were given water containing 0.025% Phenobarbital (PB) tumor promoter for the duration of the experiment. To induce expression of the Mx-Cre transgene and cause deletion of the Foxm1 f1/f1 allele in preexisting liver tumors, the mice were injected three times (each one day apart) with 250 μg of synthetic double stranded RNA (dsRNA) polyinosinic-polycytidylic acid (poly(1-C); Sigma-Aldrich, St. Louis, Mo.). The PB administration was continued in the drinking water to allow tumor growth.

Cell penetrating WT (D-ARG)₉-ARF 26-44 peptide (rrrrrrrrrKFVRSRRPRTASCALAFVN; SEQ ID NO: 3) and mutant (D-ARG)₉-ARF 37-44 peptide (rrrrrrrrrSCALAFVN; SEQ ID NO: 6) were synthesized by Genemed Synthesis, Inc. (South San Francisco, Calif.). Foxm1 f1/f1 mice with hepatic tumors induced by 32 weeks of DEN/PB exposure as discussed above were subjected to daily IP injections of 5 mg/Kg body weight of the WT (D-ARG)₉-ARF 26-44 peptide or mutant (D-ARG)₉-ARF 37-44 peptide for 4 weeks and with WT (D-ARG)₉-ARF 26-44 peptide for 8 weeks. Tumor bearing mice were also injected with sterile phosphate buffered saline (PBS) as a control. Mice were sacrificed by CO₂ asphyxiation. Livers from sacrificed mice were dissected and paraffin embedded for immunostaining and for isolation of protein extracts.

Liver sections were immunostained with anti-survivin antibodies (Novus Biologicals, Littleton, Colo.) or anti-CD34 antibodies (RAM34, BD Biosciences, San Jose, Calif.). Liver extracts were subjected to Western blot analysis with anti-survivin antibodies, anti-PUMA antibodies (Cell Signaling, Beverly, Mass.), and anti-nucleophosmin antibodies (anti-NPM/B23; Zymed, San Francisco, Calif.). Anti-β-actin was used as a loading control.

Angiogenesis is critical to mediating HCC (hepatic hepatocellular carcinoma) growth, and the endothelial cells of new HCC capillaries exhibit expression of the CD34 protein. Abundant CD34 staining was found in endothelial cells of HCC regions in PBS or mutant ARF 37-44 peptide treated mice (FIGS. 2A-2B) and from dsRNA (CKO) Mx-Cre Foxm1 −/− mice (FIG. 2D). In contrast, expression of CD34 protein is extinguished in endothelial cells from WT ARF 26-44 peptide treated mouse HCC (FIG. 2C).

These results suggest that WT ARF 26-44 peptide treatment was preventing HCC angiogenesis, which was likely caused by apoptosis of new HCC endothelial cells (See FIG. 8B, small apoptotic cells). In order to determine whether WT ARF 26-44 peptide induces apoptosis of endothelial cells, human microvascular endothelial cells (HMEC-1 cells) were treated for 48 hours with 100 μM of WT ARF26-44 peptide or mutant ARF 37-44 peptide or with PBS and then assayed for apoptosis by the terminal deoxynucleotidyl transferase-mediated dUTP-biotin Nick End Labeling (TUNEL) assay. The results indicate that WT ARF26-44 peptide treatment induced a significant increase in apoptosis of HMEC-1 cells compared with treatment with mutant ARF37-44 peptide or PBS (FIG. 2E). The results suggest that WT ARF 26-44 peptide is able to induce apoptosis of endothelial cells, which contributes to WT ARF peptide-mediated reduction in HCC angiogenesis.

Additionally, Mutant ARF 37-44 peptide and PBS treated liver tumors displayed abundant nuclear and cytoplasmic staining of survivin protein (FIGS. 2F-2G). Survivin is overexpressed in tumor cells to prevent apoptosis. Nuclear levels of survivin were diminished in HCC regions from the WT ARF 26-44 peptide treated and dsRNA CKO Mx-Cre Foxm1 −/− mice (FIGS. 2H-2I).

Western blot analysis showed that Foxm1 −/− liver tumors displayed a 60% decrease in expression of survivin protein (FIG. 2J) and no apoptosis was detected in these Foxm1 deficient liver tumors as observed by staining with hemotoxylin and eosin. (FIGS. 3E-3G) In contrast, a 90% decrease in survivin protein levels was found in hepatic tumors from WT ARF 26-44 peptide treated mice (FIGS. 2H-2J), which correlated with significant levels of apoptosis as determined by the TUNEL assay on liver sections using ApoTag Fluorescein in situ apoptosis detection kit from Intergen (Purhcase, N.Y.). (FIG. 8B) These results demonstrated that WT ARF 26-44 peptide treatment induces apoptosis of HCC by bringing in vivo levels of survivin protein below a critical threshold. Western blot analysis also showed that ARF 26-44 peptide treatment did not significantly alter levels of nucleolar mucleophosmin (NPM/B23) protein or PUMA, indicating that the HCC apoptosis did not involve the p53-PUMA pathway (FIG. 2K).

The results of these experiments suggest that WT ARF 26-44 peptide treatment prevented HCC angiogenesis by inducing HCC endothelial cell apoptosis.

### Example 2

### The Mouse Foxm1 Transcription Factor is Required for Hepatic Tumor Progression

In order to determine whether or not Foxm1 is required for hepatic tumor progression, the Interferon α/β regulated Mx-Cre recombinase (Mx-Cre) transgene (Kuhn et al., 1995, Science 269:1427-1429) was used to conditionally knockout (CKO) or delete the Foxm1 f1/f1 targeted allele in preexisting liver tumors induced by the DEN/PB exposure as previously described (Kalinichenko et al., 2004, Genes & Development 18:830-850). Hepatocellular carcinomas (HCC) were induced in mice with 30 weeks of Diethylnitrosamine (DEN)/Phenobarbital (PB) exposure, and then induced Mx-Cre expression with synthetic double stranded RNA (dsRNA) to conditionally knock out (CKO) the Foxm1 f1/f1 targeted allele. Mice were then subjected to an additional 10 weeks of PB tumor promotion protocol (FIG. 3A). To obtain long term BrdU labeling of the liver tumors, the mice were then given drinking water containing 1 mg/ml of Bromodeoxyuridine (BrdU) for 4 days (Kalinichenko et al., 2004, Genes & Development 18:830-850; Ledda-Columbano et al., 2002, Hepatology 36:1098-1105). The Mx-Cre transgene efficiently deleted the Foxm1 f1/f1 targeted allele as evidenced by the absence of detectable nuclear staining of Foxm1 protein in liver tumors of dsRNA CKO Mx-Cre Foxm1 −/− mice compared to control liver tumors (FIGS. 3B-3D).

Liver sections stained with Hematoxylin and Eosin (H&E) were used to determine the number of tumors per cm² of liver tissue (FIGS. 3E-3G). Micrographs of H&E stained liver tumor sections taken by an Axioplan2 microscope (Carl Zeiss) and the Axiovision program (Version 4.3; Carl Zeiss) were examined to calculate the area or size of liver tumors. After 40 weeks of DEN/PB exposure, control mice displayed hepatic adenomas and HCC that were larger than 2 mm² in size (Table 1).

TABLE 1
WT ARF peptide treatment diminishes number and size
of hepatic adenomas and HCC per cm² liver tissue:

^{A}Foxm1 Mouse

| Genotype or ARF | | ^{C}No. of liver tumors | | ^{D}No. of liver tumors | |
|---|---|---|---|---|---|
| peptide treatment | ^{B}No. | between 0.1 and 2.0 mm² | | greater than 2.0 mm² | |
| 40 wks DEN/PB | mice | No. Ad. | No. HCC | No. Ad. | No. HCC |
| dsRNA (Control) | 6 | 2.8 ± 1.8 | 7.1 ± 4.0 | 2.6 ± 1.3 | 4.7 ± 1.3 |
| Foxm1 fl/fl | | | | | |
| PBS (Control) | 5 | 1.3 ± 0.7 | 9.2 ± 4.6 | 3.9 ± 1.5 | 2.1 ± 1.1 |
| Mx-Cre Foxm1 fl/fl | | | | | |
| dsRNA (CKO) | 6 | 2.2 ± 1.7 | ^{E}*3.0 ± 1.1 | *0.22 ± 0.4 | **0.2 ± 0.4 |
| Mx-Cre Foxm1 —/— | | | | | |
| (Foxm1 inhibitor) WT ARF | 5 | *1.6 ± 0.6 | **3.0 ± 2.1 | **2.1 ± 0.8 | 0 |
| 26-44 Peptide Treatment | | | | | |
| (Control) Mutant ARF | 4 | 4.9 ± 1.5 | 11.7 ± 2.7 | 4.5 ± 0.9 | 4.9 ± 1.4 |
| 37-44 Peptide Treatment | | | | | |

^{A}See examples for details of conditional deletion of Foxm1 fl/fl targeted allele and for induction of hepatic tumors in response to Diethylnitrosamine (DEN)/Phenobarbital (PB) exposure and treatment cell penetrating WT (ARG)₉ ARF 24-44 (WT ARF 26-44) peptide or Mutant (ARG)₉ ARF 37-44 (Mutant ARF 37-44) peptide.
^{B}No. Mice: Number of male mice analyzed for liver tumors after 40 weeks of DEN/PB exposure.
^{C,D}The number of liver tumors per cm² liver tissue ± SD was determined from Hematoxylin and Eosin stained liver sections obtained from four different mouse liver lobes. Hepatic adenomas (Ad.) or hepatocellular carcinomas (HCC) found in mouse livers between 0.1 mm and 2 mm² in size^{C} or greater than 2 mm² in size^{D}.
^{E}The asterisks indicates statistically significant changes:
*P ≦ 0.05 and
**P ≦ 0.01.
Tumor size of cell penetrating WT ARF 26-44 peptide treated versus mutant ARF 37-44 peptide treated liver tumors was compared. Tumor size of dsRNA (CKO) Mx-Cre Foxm1 —/— liver tumors versus controls was also compared.

Deletion of Foxm1 in preexisting hepatic tumors in dsRNA CKO Mx-Cre Foxm1 −/− mice caused a significant reduction in the number of liver tumors larger than 2 mm² in size compared to control liver tumors after 40 weeks of DEN/PB exposure (Table 1). Tumor cell proliferation was measured by determining the number of hepatic tumor cells that immunostained positive for BrdU incorporation. Compared to control liver tumors, dsRNA CKO Mx-Cre Foxm1 −/− mice displayed an 80% reduction in the number of liver tumor cells that stained positive for BrdU after 40 weeks of DEN/PB treatment (FIGS. 3H-3K). Taken together, these results indicated that deletion of Foxm1 in preexisting liver tumors significantly diminished proliferation and growth of hepatic cancer cells.

### Example 3

### The Cell Penetrating WT ARF 26-44 Peptide Targets the Endogenous Mouse Foxm1 Protein to the Nucleolus of Hepatic Tumor Cells

A synthetic, cell penetrating ARF 26-44 peptide fused to 9 N-terminal D-Arg residues (Fuchs et al., 2000, Biochemistry. 43:2438-2444; Wender et al., 2000, Proc Natl Acad Sci USA 97:13003-13008), was efficiently transduced into osteosarcoma U20S cells and inhibited FoxM1b transcriptional activity as described in (Kalinichenko et al., 2004, Genes & Development 18:830-850). Treatment of U20S cells with 12 μM of the tetramethylrhodamine (TMR) fluorescently tagged (D-ARG)₉-ARF 26-44 (WT ARF 26-44, SEQ ID NO:3) peptide targeted nuclear GFP-FoxM1b fusion protein to the nucleolus (FIGS. 4B-4C) and co-localized with WT ARF 26-44 peptide fluorescence (FIG. 4C-4D). In contrast, GFP-FoxM1b protein remained nuclear in U20S cells when treated with a TMR fluorescently tagged mutant (D-ARG)₉-ARF 37-44 ARF (Mut. ARF 37-44, SEQ ID NO:4) peptide (FIG. 4E), which lacked the amino acids 26 to 36 required to interact with the FoxM1b protein. Because Arg-rich sequences are sufficient for nucleolar targeting, the mutant ARF 37-44 peptide fluorescence also localized to the nucleolus of U20S cells (FIG. 4F). No signal was observed in the absence of the ARF-peptide.

In order to determine the effective concentration of dose of the ARF peptide for efficient liver delivery, mice were subjected to IP injection of either 0.1, 1, 5 or 10 mg/Kg body weight of TMR fluorescently tagged WT ARF 26-44 peptide, and were sacrificed 24 hours later, after which their livers were dissected, formalin fixed and paraffin embedded. Liver sections were treated with Xylene to remove paraffin wax and then examined by fluorescent microscopy for red peptide fluorescence. This dose response curve determined that IP injection of either equal or greater than 5 mg/Kg body weight of TMR-fluorescently labeled WT ARF 26-44 peptide was detectable in cytoplasm and nucleolus of hepatocytes and in hepatic mesenchymal cells at 24 hours after injection (FIG. 4G). Based on these studies, hepatic tumors were induced in Foxm1 f1/f1 mice by 32 weeks of DEN/PB exposure and then they were subjected to daily IP injections of 5 mg/Kg body weight of the cell penetrating WT ARF 26-44 peptide or Mutant ARF 37-44 peptide for 4 weeks and with WT ARF 26-44 peptide for 8 weeks (FIG. 4A). After 33 weeks of DEN/PB treatment, ARF −/− Rosa26-FoxM1b TG mice were subjected to daily IP injections of 5 mg/Kg body weight of the cell penetrating WT ARF 26-44 peptide or Mutant ARF 37-44 peptide for 4 weeks. Liver tumor bearing mice were also administered sterile PBS as controls.

After 4 weeks of treatment with TMR fluorescently labeled ARF peptides, laser confocal microscopy of paraffin embedded mouse liver tumor sections revealed that ARF peptide fluorescence localized to the hepatocyte cytoplasm and nucleolus (FIGS. 4H-4I) and was uniformly distributed throughout the liver parenchyma. The Foxm1 protein staining in WT ARF 26-44 peptide treated liver tumor sections was partially localized to the nucleolus in hepatic tumor cells (FIG. 4L; black arrows), which was similar to the immunostaining pattern of the nucleolar protein nucleophosmin (FIG. 4J; NPM; black arrows). In contrast, mutant ARF 37-44 peptide or PBS treated liver tumor cells displayed only nuclear Foxm1 staining (FIGS. 4K and 4M). These studies demonstrated that the WT ARF 26-44 peptide reduces in vivo function of Foxm1 by partially targeting the endogenous Foxm1 protein to the nucleolus of hepatic tumor cells.

### Example 4

### WT ARF 26-44 Peptide Diminishes Proliferation and Size of Liver Tumors

To monitor hepatic cellular proliferation, PB was removed 4 days prior to the completion of the experiment, and mice were placed on drinking water with 1 mg/ml of 5-bromo-2-deoxyuridine (BrdU) for 4 days before they were sacrificed. Hepatic tumor cell DNA replication in liver sections was determined by immunohistochemical detection of BrdU incorporation (mouse anti-BrdU (Bu20a, 1:100; DakoCytomation). Hepatic tumor cells were examined to determine the number that incorporated BrdU in mice treated with cell penetrating WT ARF 26-44 peptide, mutant ARF 37-44 peptide or PBS. Significant reduction in BrdU incorporation was found in liver tumors that had been treated with the WT ARF 26-44 peptide for 4 or 8 weeks compared to mouse liver tumors treated with mutant ARF 37-44 peptide or PBS (FIGS. 5A through 5M). Compared to control mouse liver tumors, treatment with the WT ARF 26-44 peptide for 8 weeks significantly reduced tumor growth and prevented development of HCC larger than 2 mm² in size (Table 1). These results indicated that treatment with the WT ARF 26-44 peptide was an effective method with which to reduce proliferation and growth of hepatocellular carcinomas.

Expression and localization of p27^{Kip} was then examined in the HCC cells, because nuclear accumulation of p27^{Kip} is known to be associated with Foxm1 (−/−) hepatic tumors. The WT ARF 26-44 peptide treated HCC cells displayed increased nuclear levels of the p27^{Kip1} protein, as detected by immunohistochemistry using mouse anti-Kip1/p27 antibodies (1:100; BD Biosciences), which was similar to those found with dsRNA CKO Mx-Cre Foxm1 −/− liver tumors (FIGS. 6B and 6E). In contrast, p27^{Kip1} immunostaining was predominantly cytoplasmic in mutant ARF 37-44 peptide or PBS treated mouse HCC (FIGS. 6A, 6C, 6D and 6F). These studies indicated that the WT ARF 26-44 peptide was effective in reducing Foxm1 function in vivo and that nuclear accumulation of p27^{Kip1} protein was associated with reduced hepatic tumor proliferation.

### Example 5

### WT ARF 26-44 Peptide Causes Selective Apoptosis of Hepatic Tumor Cells

Analysis of H&E stained liver tumor sections from mice treated with the WT ARF 26-44 peptide revealed that many of the hepatic adenomas and HCC tumor cells stained red and exhibited disruption of nuclear membrane, which was indicative of apoptosis (FIGS. 7A-7F). The red staining cells were found neither in the surrounding normal liver tissue (FIGS. 7A-7F) nor in hepatic tumors from mice treated with either the mutant ARF 37-44 peptide or PBS (FIGS. 7G-7L). Furthermore, these apoptotic tumor cells were not apparent in FoxM1 deficient livers in dsRNA (CKO) Mx-Cre Foxm1−/− mice (FIG. 3E-3G).

To measure apoptosis in mouse livers we used the Terminal Deoxynucleotidyl Transferase-mediated dUTP-biotin Nick End Labeling (TUNEL) assay on liver sections using the ApoTag Fluorescein in situ apoptosis detection kit from Intergen (Purchase, N.Y.) according to the manufacturer's recommendations. The mean number (±SD) of TUNEL- or DAPI-positive hepatocyte nuclei was calculated per 1000 cells or 200× field by counting the number of positive hepatocyte nuclei using five different 200× fields of liver tumor sections from male mice at the indicated times of DEN/PB exposure. The TUNEL assay showed that mouse HCC cells treated with WT ARF 26-44 peptide exhibited a significant 22% increase in apoptosis (FIGS. 8A-8B and 8E). In contrast, very few apoptotic HCC cells were found after treatment with mutant ARF 37-44 peptide or PBS (FIGS. 8 C-8E). Immunostaining of liver tumor sections with proteolytically cleaved activated caspase 3 protein confirmed this selective apoptosis of mouse HCC cells treated with WT ARF 26-44 peptide with no pro-apoptotic staining in the adjacent normal liver tissue (FIGS. 8F-8H). These studies showed that the WT ARF peptide selectively induced apoptosis of HCC cells without damaging adjacent normal hepatocytes.

### Example 6

### Den/PB treatment induced highly proliferative HCC in ARF −/− Rosa26 FoxM1b Transgenic Mice that are Responsive to WT ARF 26-44 Peptide Treatment

In order to develop a new genetic model of HCC that is highly dependent on FoxM1b transcription factor, Rosa26-FoxM1b TG mice were crossed into the ARF −/− mouse background, which overexpressed FoxM1b and eliminated ARF inhibition of FoxM1 transcriptional activity. After 33 weeks of DEN/PB treatment, ARF −/− Rosa 26 FoxM1b TG mice developed highly proliferative HCC and their HCC cells displayed a proliferation rate of 6000 Bromodeoxyuridine (BrdU) positive cells per mm² tumor (FIG. 9J), which is approximately 30-times greater than that observed in DEN/PB induced HCC in WT mice (FIG. 5K, 200 BrdU positive cells per mm² tumor). The DEN/PB treated ARF −/− Rosa 26 FoxM1b TG livers also exhibited development of necrosis and fibrosis/cirrhosis.

These HCC-tumor bearing ARF −/− Rosa 26 FoxM1b TG mice were subjected to daily treatment with either the cell penetrating WT ARF 26-44 peptide or Mutant ARF 37-44 peptide for 4 weeks. In ARF −/− Rosa 26 FoxM1b TG mice, WT ARF 26-44 peptide treatment resulted in a significant 84% reduction in Bromodeoxyuridine (BrdU) labeling of HCC cells compared to treatment of these mice with either Mutant ARF 37-44 peptide or PBS (FIGS. 9A-9C and 9J). Red staining HCC cells with disruption of nuclear membrane indicative of apoptosis were found in H&E stained liver tumor sections from ARF −/− Rosa 26 FoxM1b TG mice treated with the WT ARF 26-44 peptide but not in those treated with mutant ARF 37-44 peptide or PBS (FIGS. 9D-9F). A TUNEL assay was then conducted, demonstrating that ARF −/− Rosa 26 FoxM1b TG mice HCC cells treated with WT ARF 26-44 peptide exhibited a 42% increase in apoptosis (FIG. 9K), which is twice as high as in liver tumors from wild type mice (FIG. 8E). Furthermore, TUNEL-positive cells were restricted to the HCC region (white arrow heads) and were not detected in adjacent normal liver tissue (FIG. 91). In contrast, very few apoptotic HCC cells were found after treatment of ARF −/− Rosa 26 FoxM1b TG mice with mutant ARF 37-44 peptide or PBS (FIGS. 9G-9H and 9K). These ARF −/− Rosa 26 FoxM1b TG liver tumor studies showed that the cell penetrating WT ARF 26-44 peptide was effective in diminishing BrdU labeling of highly proliferative HCC cells and selectively induced apoptosis of HCC cells in these mice without damaging adjacent normal liver tissue.

### Example 7

### WT ARF Peptide Induced Apoptosis of Human Hepatoma HepG2 Cells Correlates with Diminished Expression of Survivin, PLK1 and Aurora B Kinase

HepG2 cells were electroporated with 100 nM of FoxM1 (FoxM1 #2) or p27^{Kip1} (siP27) siRNA duplexes (Wang et al., 2005, Mol Cell Biol 25:10875-10894) using the Nucleofector™ II apparatus (Amaxa Biosystems, Gaithersburg, Md.) and eletroporation buffers recommended by the manufacturer for HepG2 cells. HepG2 cells were replated for two days to allow siRNA silencing of FoxM1 or p27Kip1 levels and then 2×10⁵ HepG2 cells were plated in triplicate and viable HepG2 cells were counted at 2, 3, 4 or 5 days following electroporation. Mock electroporated cells were used as controls. Also, 2×10⁵ HepG2 cells were plated in triplicate and viable HepG2 cells were counted at 1, 2 or 3 days following treatment with 50 μM of WT ARF 26-44 peptide or Mutant ARF 37-44 peptide. After two days in culture, media was replaced with 50 μM of WT ARF 26-44 peptide or Mutant ARF 37-44 peptide. PBS treated cells were used as controls.

A TUNEL assay was conducted as described above, which revealed that human hepatoma HepG2 cells (FIG. 10A-10E), PLC/PRF/5 cells that express mutant p53 protein and p53 deficient Hep3B cells exhibited 50% apoptosis after 24 hours of treatment with 25 μM of WT ARF 26-44 peptide (FIG. 10E), whereas only low levels of apoptosis were detected in these cells following treatment with mutant ARF 37-44 peptide or PBS (FIG. 10E). Diminished levels of p53 protein through p53 siRNA silencing of HepG2 cells did not influence apoptosis in response to WT ARF 26-44 peptide treatment (FIG. 10F). In addition, p53 protein levels were unaltered in HepG2 cells after 24 hours of treatment with WT ARF 26-44 (WT) or mutant ARF 37-44 (Mutant) peptide (FIG. 10F). Furthermore, protein expression of the p53 downstream pro-apoptotic target PUMA was unchanged in HepG2 cells in response to increasing concentrations of the WT ARF 26-44 peptide (FIG. 10). These results demonstrated that WT ARF 26-44 peptide-induced apoptosis was independent of the p53-PUMA pro-apoptotic pathway. Moreover, HepG2 cells depleted in FoxM1 levels by electroporation of FoxM1 no. 2 siRNA duplexes were resistant to apoptosis in response to WT ARF 26-44 peptide treatment (FIG. 10F), suggesting that induction of apoptosis by the WT ARF peptide was dependent on FoxM1 levels.

Tumor cells are known to express high levels of the mitotic regulators polo-like kinase 1 (PLK1), Aurora kinase and survivin proteins, where they function to prevent apoptosis of cancer cells, and previous studies demonstrated that U20S cells transfected with siFoxM1 #2 duplex were blocked in mitotic progression and exhibited undetectable levels of FoxM1 and its downstream target mitotic regulators PLK1, aurora B kinase and survivin (Wang et al., 2005, Mol Cell Biol 25:10875-10894). Consistent with these studies, FoxM1 depleted HepG2 cells exhibited undetectable protein levels of survivin, PLK1 and aurora B kinase (FIG. 10G). HepG2 cells were electroporated with siFoxM1 #2 or control p27^{Kip1} siRNA (siP27), and the cells were grown in culture for two days to allow for siRNA silencing. 2×10⁵ HepG2 cells were then plated in triplicate and viable HepG2 cells were counted at 2, 3, 4 or 5 days following electroporation. These cell growth studies showed that FoxM1 deficient HepG2 cells were unable to grow in culture and gradually detached from the plate with time in culture (FIG. 10H). In contrast, HepG2 cells treated with WT ARF 26-44 peptide exhibited a less severe reduction in levels of survivin (50%), PLK1 (80%) and aurora B kinase (80%) proteins compared to controls (FIG. 10). The growth curve of HepG2 cells at 1, 2 or 3 days following treatment with WT ARF 26-44 peptide, Mutant ARF 37-44 peptide or PBS was also determined. Although the WT ARF 26-44 peptide treated HepG2 cells displayed 50% apoptosis (FIG. 10E), they were able to sustain the number of cells initially plated (2×10⁵), suggesting that the WT ARF peptide treated cells were able to proceed through the cell cycle (FIG. 10J). These results were consistent with recent studies in which hypomorphic levels of FoxM1 protein (40% of WT FoxM1 levels) in breast cancer cell lines transfected with a different FoxM1 siRNA duplex reduced expression of mitotic regulators to levels that are insufficient to properly execute mitosis, leading to mitotic catastrophe and apoptosis (Wonsey et al., 2005, Cancer Res 65:5181-5189). Thus, these studies provide evidence that WT ARF 26-44 peptide treatment causes hypomorphic levels of Foxm1 activity, leading to apoptosis, whereas depleting Foxm1 levels results in mitotic arrest.

It should be understood that the foregoing disclosure emphasizes certain specific embodiments of the invention and that all modifications or alternatives equivalent thereto are within the spirit and scope of the invention as set forth in the appended claims.

What is claimed is:

1. A method for inhibiting angiogenesis in a mammal, said method comprising administering to the mammal an effective amount of a peptide having an amino acid sequence identified by SEQ ID NO: 4.

2. The method of claim 1, wherein the peptide is covalently linked to a cell-penetrating molecule.

3. The method of claim 2, wherein the cell-penetrating molecule has an amino acid sequence identified by SEQ ID NO: 10.

4. The method of claim 3, wherein the peptide has an amino acid sequence identified by SEQ ID NO:3.

5. The method of claim 1, wherein the mammal has a solid tumor.

6. The method of claim 1, wherein angiogenesis is inhibited in a non-cancerous tissue in the mammal.

7. The method of claim 1 wherein the peptide has the amino acid sequence identified by SEQ ID NO:3 or SEQ ID NO:4.

8. The method of claim 7, wherein the peptide has the amino acid sequence identified by SEQ ID NO:3.

9. A method for inhibiting in a mammal a biological process comprising angiogenesis, said method comprising administering to the mammal an effective amount of a peptide having an amino acid sequence identified by SEQ ID NO: 4.

10. The method of claim 9, wherein the peptide is covalently linked to a cell-penetrating molecule.

11. The method of claim 10, wherein the cell-penetrating molecule has an amino acid sequence identified by SEQ ID NO:10.

12. The method of claim 11, wherein the peptide has an amino acid sequence identified by SEQ ID NO:3.

13. The method of claim 9 wherein the peptide has the amino acid sequence identified by SEQ ID NO:3 or SEQ ID NO:4.

14. The method of claim 13, wherein the peptide has the amino acid sequence identified by SEQ ID NO:3.

15. The method of claim 9 wherein the biological process is selected from the group consisting of angiogenic factor production, angiogenic factor release, endothelial cell receptor binding, endothelial cell activation, endothelial cell migration, endothelial cell proliferation, extracellular matrix (ECM) remodeling, tube formation, formation of new blood vessels from existing blood vessels, and vascular stabilization.

16. The method of claim 15 wherein the biological process is endothelial cell proliferation.

17. A method for inhibiting an angiogenesis-related disease in a mammal, said method comprising administering to the mammal a peptide having an amino acid sequence identified by SEQ ID NO:4.

18. The method of claim 17, wherein the peptide is covalently linked to a cell-penetrating molecule.

19. The method of claim 18, wherein the cell-penetrating molecule has an amino acid sequence identified by SEQ ID NO:10.

20. The method of claim 19, wherein the peptide has an amino acid sequence identified by SEQ ID NO:3.

21. The method of claim 17 wherein the peptide has the amino acid sequence identified by SEQ ID NO:3 or SEQ ID NO:4.

22. The method of claim 21, wherein the peptide has the amino acid sequence identified by SEQ ID NO:3.

23. The method of claim 17, wherein the angiogenesis-related disease is selected from the group consisting of immune and non-immune inflammation, rheumatoid arthritis, chronic articular rheumatism, psoriasis, diabetic retinopathy, neovascular glaucoma, retinopathy of prematurity, macular degeneration, loss of vision due to invasion of blood vessel, corneal graft rejection, retrolental fibroplasia, rubeosis, capillary proliferation in atherosclerotic plaques, osteoporosis, solid tumors, tumor metastases, leukemias, angiofibromas, Kaposi sarcoma, hemangiomas, acoustic neuromas, neurofibromas, trachomas, pyogenic granulomas, Osler-Webber Syndrome, myocardial angiogenesis, plaque neovascularization, telangiectasia, edema, hemophiliac joints, and wound granulation.

24. The method of claim 23, wherein the angiogenesis-related disease is tumor.

25. The method of claim 24, wherein the tumor is liver tumor.

26. The method of claim 25, wherein the liver tumor is hepatocellular carcinoma.

27. The method of claim 25, wherein the liver tumor is hepatic adenoma.

28. A pharmaceutical composition for inhibiting angiogenesis, said composition comprising a therapeutically effective amount of a peptide having an amino acid sequence identified by SEQ ID NO:4.

29. The pharmaceutical composition of claim 28, wherein the peptide is covalently linked to a cell-penetrating molecule.

30. The pharmaceutical composition of claim 29, wherein the cell-penetrating molecule has an amino acid sequence identified by SEQ ID NO:10.

31. The pharmaceutical composition of claim 30, wherein the peptide has an amino acid sequence identified by SEQ ID NO:3.

32. The pharmaceutical composition of claim 28 wherein the peptide has the amino acid sequence identified by SEQ ID NO:3 or SEQ ID NO:4.

33. The pharmaceutical composition of claim 32, wherein the peptide has the amino acid sequence identified by SEQ ID NO:3.
